(19) United States
(12) Patent Application Publication
(10) Pub. No.: US 2008/0193554 A1
(43) Pub. Date: Aug. 14, 2008

(54) Surgical Membrane

(75) Inventors: Harminder Dua, Nottingham (GB); Andrew Hopkinson, Nottingham (GB)

Correspondence Address: DAVIS WRIGHT TREMAINE LLP/Los Angeles, 865 FIGUEROA STREET, SUITE 2400, LOS ANGELES, CA, 90017-2566, US

(73) Assignee: THE UNIVERSITY OF NOTTINGHAM, Nottingham (GB)

(21) Appl. No.: 11/995,579

(22) PCT Filed: Jul. 17, 2006
(86) PCT No.: PCT/GB2006/050209

§ 371 Date: Jan. 14, 2008

(30) Foreign Application Priority Data
Jul. 15, 2005 (GB) 0514567.7

(51) Int. Cl. A61K 35/23; A01N 1/02; A61P 43/00

(52) U.S. Cl. 424/558; 435/1.1; 435/1.3

(57) ABSTRACT

The invention relates to methods of processing amniotic membrane to generate a substantially ‘growth factor free’ membrane (GFF-membrane), and to methods of processing GFF-membrane to generate membrane enriched with specific and quantified levels of growth factors or other desirable membrane enriching molecules or compounds (E-membrane). The invention extends to GFF-membrane per se and E-membrane enriched with specific and quantified membrane-enriching compounds per se. The method also includes first and second medical uses of GFF-membrane and E-membrane. The method also extends to clinical uses of the amniotic membrane spongy layer or components thereof.

The invention relates to methods of processing amniotic membrane to generate a substantially ‘growth factor free’ membrane (GFF-membrane), and to GFF-membrane. The invention extends to methods of processing GFF-membrane to generate membrane enriched with specific and quantified levels of growth factors or other desirable membrane enriching molecules or compounds (E-membrane), and to E-membrane enriched with specific and quantified membrane-enriching compounds. The method also includes first and second medical uses of GFF-membrane and E-membrane. The method also extends to clinical uses of the amniotic membrane spongy layer or components thereof.

The use of foetal membranes as surgical material in skin transplantation was first reported in 1910 by Davis [1]. Surgical use of amniotic membranes has increased significantly since that first report. It is now used as a biological dressing for burned skin, skin wounds, and chronic ulcers of the leg; as an adjunctive tissue in surgical reconstruction of artificial vagina, and for repairing omphaloceles; to prevent tissue adhesion in surgical procedures of the abdomen, head, and pelvis [2-21]. Several authors reported its use in treating a variety of ocular surface disorders in the 1940s [4, 11, 22] but its use was abandoned until recently [1990s], when it was reintroduced to ophthalmologists.

Certain characteristics make the amniotic membrane ideally suited to its application in ocular surface reconstruction and the scope of the application of amniotic membrane transplantation (AMT) in the management of ocular surface disorders has increased considerably. The tissue can be preserved at −80° C. for several months, allowing sufficient time for virology testing, to plan surgery or consider a trial of other options. Amniotic membrane does not express HLA-A, B, or DR antigens and hence immunological rejection after its transplantation does not occur [23-25].

For normal proliferation and differentiation of corneal epithelial cells, the presence of a normal substrate or basement membrane is essential, facilitating the migration of epithelial cells [26, 27]; reinforcing adhesion of basal epithelial cells [28]; promoting epithelial differentiation [29,30]; preventing epithelial apoptosis [31]. A transplanted amniotic membrane serves as a basement membrane and promotes epithelialisation.

A number of expressed growth factors (EGF, TGF-α, KGF, HGF, bFGF and TGF-β1, -β2, -β3) are reported to effect epithelialisation [32]. The amniotic membrane produces various of these growth factors such as bFGF, HGF, and TGFβ, that can stimulate epithelialisation [33, 34], although cryopreservation of amniotic membrane may result in a decrease of growth factors and/or their activity [34].

There are several factors thought to be involved in the antifibrotic effect of the amniotic membrane [35-37], including the induction of a down-regulation of transforming growth factor β signalling, responsible for fibroblastic activation in wound healing. It is also possible that the amniotic membrane may also function as an anatomical barrier [38], keeping potentially adhesive surfaces apart. Furthermore, the avascular nature of the stroma of the amniotic membrane is believed to inhibit the incursion of new vessels.

Several methods for the preparation of amniotic membranes for surgery have been described (see Dua et al., 1999, Br. J. Opthalmol. 83: 748-52 for a review [38]). These include alcohol dehydration, preservation in antibiotics in saline, either with or without separation of the amniotic and chorionic layers. Other methods have been disclosed more recently. For example, Tseng (U.S. Pat. Nos. 6,152,142 and 6,326,019B1) discloses a method relying on freezing for preservation of the amniotic membrane; Hariri et al (US Patent 20040048796) disclose a method designed to decellularise the amniotic membrane leaving it devoid of all but a collagenous membrane. However, all of these methods have failings, such as irreproduciblity; they are time consuming; they are damaging to the membrane; and they result in frequently generating a fragile membrane.

Furthermore, despite many useful properties of the amniotic membrane, inconsistencies in the clinical outcome following the use of AMT in the treatment of certain conditions have been observed within the inventors' clinical practice, including excessive scarring in severe conjunctival wounds. Furthermore, although AM is an effective temporary skin replacement, it is not always available on demand and its cumbersome unreliable retrieval, coupled with the need for cleansing and sterilisation, deter its use by surgeons.

Thus, there is a need in the art for an amniotic membrane with the capability to act as a basement membrane for re-epithialisation without the induction of scarring, which demonstrates more reliable clinical benefits and is easy to prepare and store in a reliable format. Therefore, it is an object of the present invention to provide defined processes for the production of a ‘substantially growth factor free’ membrane suitable for direct surgical use and an ‘enriched’ membrane containing controlled quantities of desirable membrane-enriching compounds, for example, specific growth factor/s.

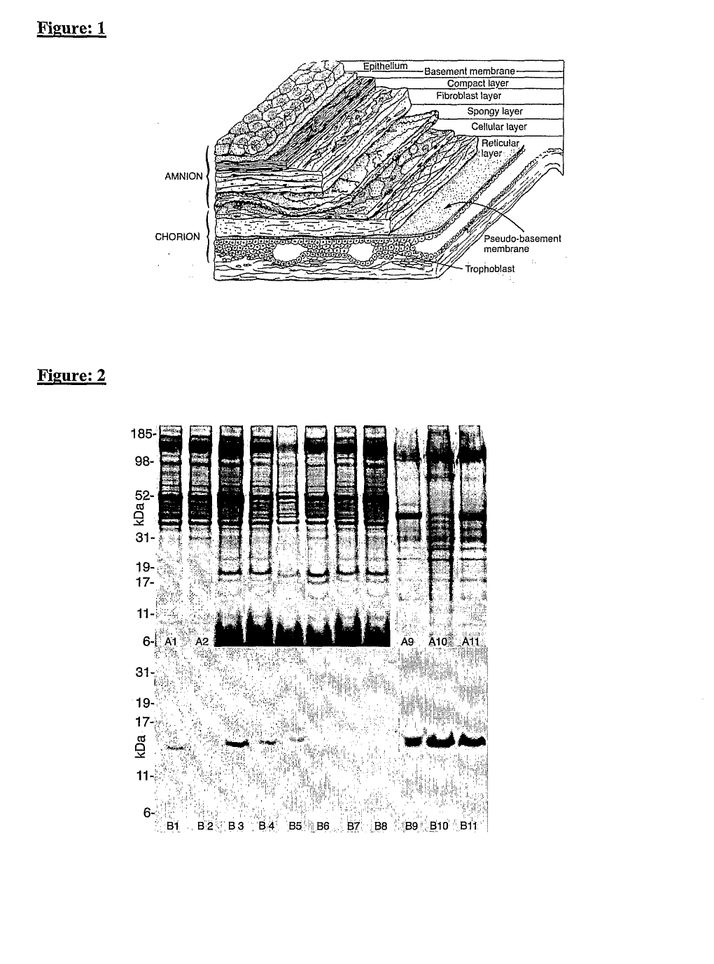

As will be described in the Examples below, the present inventors have surprisingly found that the mucinous acellular ‘spongy’ layer of the amniotic membrane (see FIG. 1) contains high levels of growth factors, being a major repository of a multitude of proteins. It is the inventors' belief that inter-donor variation, and inconsistent preparation, preservation and processing procedures result in variation in the protein composition of AMs, leading to variation in the clinical efficacy of the procedure and the degree of scarring. More specifically, they propose that the uncontrolled and inconsistent level of such growth factors present in the spongy later, as well as their preservation in active form or probable decrease and possible inactivation during cryo-preservation, may lead to inconsistencies in the clinical outcome of AMT treatment, including the risk of excessive scarring in the former situation.

The inventors have therefore devised a method for the removal of this spongy layer, before preservation of the membrane, leaving a novel membrane, being substantially soluble growth factor free, referred to herein as GFF-membrane.

Therefore, according to a first aspect of the present invention, there is provided a method of preparing substantially growth factor free amniotic membrane (GFF-membrane), which method comprises the steps of:

a) isolating amniochorionic membrane from placenta; and

b) removing a chorion membrane and spongy layer from the amniochorionic membrane, to thereby produce substantially growth factor free (GFF) amniotic membrane.

By the term “amniochorionic membrane”, we mean the combined membrane formed from the amniotic membrane and the chorion membrane.

The method according to the first aspect of the invention involves processing amniochorionic membrane in order to remove at least the growth factor rich ‘spongy’ layer, and preferably, as an intact layer. Advantageously, the method ensures that there is no need for any mechanical, chemical, abrasive or any harsh mechanism of dissociation of the spongy layer from the amniochorionic membrane, thereby leaving the underlying architecture of the amniotic membrane intact, undamaged and free from any potential chemical contamination. This is unlike current methods which do not attempt to remove this spongy layer at all, and therefore suffer the problem that they contain undefined, high concentrations of various growth factors, such as TGFβ, and therefore cause excessive scarring when use to treat wounds in patients. Hence, advantages of the method of the invention are that the GFF amniotic membrane prepared by the method is substantially devoid of all soluble growth factors, which would have a detrimental effect when use in medical treatment regimes, for example, causing excessive scarring. Hence, the GFF amniotic membrane prepared by the method has the capability to act as a basement membrane for re-epithialisation in wounds without the induction of scarring, which demonstrates more reliable clinical benefits and is easy to prepare and subsequently store in a reliable format.

It will be appreciated that the substantially growth factor free (GFF) amniotic membrane produced by the method comprises very low concentrations of growth factors, and most preferably, low concentrations of soluble growth factors.

Hence, by the term “substantially growth factor free amniotic membrane”, we mean amniotic membrane which is devoid of at least 51% (w/w) growth factors when compared to normal amniotic membrane containing the spongy layer.

Suitably, the method reduces the concentration of growth factors in the amniotic membrane by at least 55% (w/w), more suitably at least 65% (w/w), and even more suitably, at least 70% (w/w). It is preferred that the method reduces the concentration of growth factors in the amniotic membrane by at least 75% (w/w), and more preferably, at least 80% (w/w). Surprisingly, the method is very effective in producing a substantially growth factor free (GFF) amniotic membrane, i.e. at least 55% (w/w) reduction when compared to normal amniotic membrane containing the spongy layer. Figures FIG. 4.1 and 4.2 illustrate the efficacy of the method for reducing the concentration of growth factors in amniotic membrane.

The skilled technician will appreciate how to determine the concentration of growth factors in the amniotic membrane using standard isolation techniques, and Western blotting Hence, preferably, the total concentration of growth factor present in the GFF amniotic membrane following the method is less than 200 ng for a load of 20 μg of total protein extracted from 150 mg of wet tissue, more preferably, below 150 ng, even more preferably, below 100 ng, and most preferably, below 75 ng for a load of 20 μg of total protein extracted from 150 mg of wet tissue.

It will be appreciated that the actual steps of the method may be performed in accordance with any tissue dissecting, tissue washing, tissue/membrane separation and tissue storage methods known per se in the art. However, a preferred method will be found by reference to Example 2, but other suitable techniques, are available.

Preferably, step (a) of the method according to the first aspect comprises isolating the amniochorionic membrane from placenta by cutting around the periphery of the placental body. The placenta may be derived from a mother having given birth to a child via caesarean section. Preferably, step (a) is carried out no more than 15 minutes thereafter. The inventors have found that delay of longer than 15 minutes results in the isolation of inferior samples of amniochorionic membrane, and hence, amniotic membrane derived therefrom.

Preferably, after step (a) but before step (b), the method comprises a step of washing the amniochorionic membrane to remove any excess biological fluids derived from the mother, such as blood, which would otherwise be a source of considerable contamination to the resultant amniotic membrane prepared. Preferably, this washing step is conducted in sterile solution, for example, saline, which may be physiological saline. The saline solution may comprise 0.7-1.2% (w/v) NaCl, more preferably, 0.8-1% (w/v) NaCl, and most preferably, about 0.9% (w/v) NaCl. Alternatively, phosphate buffered saline, e.g. 0.1M PBS, may be used for the washing step. The washing step may be carried out for at least 10 minutes, and preferably at least 20 minutes, at room temperature, preferably with gentle shaking.

Preferably, after step (a) but before step (b), the method comprises an additional step of separating the amniochorionic membrane into amniotic membrane and chorion membrane. This chorion membrane may then be discarded or used for further analyses. The separating step may be carried out by blunt dissection through a pre-determined plane using known techniques. It is preferred that this separation step is carried out after the washing step.

Preferably, after step (a) but before step (b), the method comprises a step of soaking the amniotic membrane in a sterile solution. Preferably, soaking step comprises soaking the amniotic membrane in a solution capable of loosening the connection between various layers in the amniotic membrane, as shown in FIG. 1, for sufficient time to enable subsequent removal of the spongy layer therefrom in step (b). The spongy layer is disposed between a fibroblast layer of the amniotic membrane and a cellular layer of the chorionic membrane, and the solution used in the soaking step is preferably capable of weakening the connection therebetween. Preferably, the soaking step is carried out in saline, which may be physiological saline or phosphate buffered saline (i.e. PBS). The saline solution may comprise 0.7-1.2% (w/v) NaCl, more preferably, 0.8-1% (w/v) NaCl, and most preferably, about 0.9% (w/v) NaCl. The spongy layer is hygroscopic, therefore when it is soaked in saline, the layer swells at least 3 times its normal thickness. The soaking step may be carried out for at least 10 minutes, but preferably, at least 20 minutes, and more preferably at least 30 minutes. Preferably, the soaking step is carried out several times (ideally, at least three times) until blood contamination has been eliminated. Preferably, the soaking step is carried out at room temperature in order to maintain biological function of the amniotic membrane and also the spongy layer, which may be used itself, as will be described hereinafter.

Preferably, step (b) of the method comprises use of a scalpel to remove the spongy layer from the amniotic membrane, as described in the Example. The soaked amniotic membrane may be spread out on a flat sterile surface so that the spongy layer side of the amniotic membrane is facing upwardly. Starting in the centre, the reverse edge of a scalpel is used to apply pressure and perforate the spongy layer, but without damaging the amniotic membrane. Once a perforation front has been established across the entire piece of amniotic membrane, the scalpel and forceps may be used to gradually peel back one side of the spongy layer, preferably intact, from off the amniotic membrane. The other side of the spongy layer may then be then peeled off. Once the spongy layer has been removed, the amniotic membrane may be washed in saline to remove residual spongy layer debris for at least 20 minutes, and preferably at least 30 minutes at room temperature.

Preferably, the method of the first aspect comprises a further step after step (b), which comprises preservation and/or storage of the prepared GFF-membrane. The preservation step may comprise contacting the amniotic membrane with a suitable preservation chemical, such as, dimethyl sulfoxide (DMSO), and preferably, incubating the membrane with increasing concentrations of DMSO (for example, 4% (v/v), 8% (v/v)), and 12% (v/v)) each for a period of about 5 minutes. The amniotic membrane may then be stored in a final concentration of preservation solution, e.g. DMSO (1% v/v) in PBS, preferably, containing suitable antibiotics, for example gentamicin (160 mg/L) and/or requires a minimum of 6 months to cover the window period of infection and exclude possibility of transmission of infection. Storage will be in a freezing device at −80° C.

When ready for use, for example in surgical procedures, the method of the first aspect comprises a step of thawing the stored amniotic membrane to about room temperature, and then a washing step. The inventors have found that the preservation step causes some cells of the amniotic membrane to lyse, thereby releasing their contents, which have a tendency to stick on the cell surface. Hence, the post-preservation washing step advantageously results in the removal of contaminating biomolecules (e.g. proteins, growth factors, enzymes etc), which may be present on the thawed amniotic membrane.

Hence, preferably, after the preservation step, the method comprises an additional step of washing the amniochorionic membrane to remove cellular debris. Preferably, this washing step is conducted in sterile solution, for example, saline, which may be physiological saline. The saline solution may comprise 0.7-1.2% (w/v) NaCl, more preferably, 0.8-1% (w/v) NaCI, and most preferably, about 0.9% (w/v) NaCl. Alternatively, phosphate buffered saline, e.g. 0.1M PBS, may be used for the washing step.

The washing step may be carried out for at least 10 minutes, and preferably at least 20 minutes, at room temperature. However, preferably, the washing step comprises at least two, and preferably, at least three cycles of washes in about 50 ml (for typical amniotic membrane pieces of 4 cm×4 cm in size) saline for preferably 10 minutes each cycle. This removes the cellular debris and further reduces the levels of soluble growth factors in the amniotic membrane by at least 95%. Hence, preferably, the method including the final washing step reduces the concentration of growth factors in the amniotic membrane by at least 85% (w/w), more suitably at least 90% (w/w), and even more suitably, at least 92% (w/w), when compared to normal amniotic membrane containing the spongy layer. However, it is especially preferred that the method reduces the concentration of growth factors in the amniotic membrane by at least 95% (w/w), more preferably, at least 97% (w/w), and most preferably, at least 99% (w/w), when compared to normal amniotic membrane containing the spongy layer.

By way of example, FIG. 4.2 illustrates the concentration of TGFβ titrated from human platelets, and illustrates how much TGFβ is detected in amniotic membrane, and the spongy layer removed in step (b) of the method. Each lane of the blot shown in FIG. 4.2 represents relative TGFβ levels in 20 μg total protein extract from 150 mg amniotic membrane (wet weight) in 1 ml from which it is possible to yield about 2 mg total protein. Therefore, from the results, the inventors have demonstrated that the method of the invention surprisingly reduces TGFβ levels to below 50 ng for a load of 20 μg of total protein extracted from 150 mg of wet tissue. Hence, preferably, the total concentration of growth factor present in the GFF amniotic membrane is less than 50 ng for a load of 20 g of total protein extracted from 150 mg of wet tissue, more preferably, below 30 ng, even more preferably, below 10 ng, and most preferably, below 5 ng for a load of 20 μg of total protein extracted from 150 mg of wet tissue.

Hence, in a preferred embodiment, the method comprises the steps of:
(a) isolating amniochorionic membrane from placenta;
(b) separating the amniochorionic membrane into amniotic membrane and chorion membrane;
(c) soaking the amniotic membrane in sterile solution; and
(d) removing a growth factor rich mucinous spongy layer from the amniotic membrane, to thereby produce substantially growth factor free (GFF) amniotic membrane.

In a preferred embodiment, the method comprises the steps of:
(a) isolating amniochorionic membrane from placenta;
(b) washing the amniochorionic membrane;
(c) separating the amniochorionic membrane into amniotic membrane and chorion membrane;
(d) soaking the amniotic membrane in sterile solution; and
(e) removing a growth factor rich mucinous spongy layer from the amniotic membrane, to thereby produce substantially growth factor free (GFF) amniotic membrane.

However, in a more preferred embodiment, the method comprises the steps of:
(a) isolating amniochorionic membrane from placenta;
(b) washing the amniochorionic membrane;
(c) separating the amniochorionic membrane into amniotic membrane and chorion membrane;
(d) soaking the amniotic membrane in sterile solution;
(e) removing a growth factor rich mucinous spongy layer from the amniotic membrane, to thereby produce substantially growth factor free (GFF) amniotic membrane; and
(f) preserving the amniotic membrane.

In most preferred embodiment, the method comprises the steps of:
(a) isolating amniochorionic membrane from placenta;
(b) washing the amniochorionic membrane;
(c) separating the amniochorionic membrane into amniotic membrane and chorion membrane;
(d) soaking the amniotic membrane in sterile solution;
(e) removing a growth factor rich mucinous spongy layer from the amniotic membrane, to thereby produce substantially growth factor free (GFF) amniotic membrane;
(f) preserving the amniotic membrane; and
(g) washing the amniotic membrane.

The inventors believe that, to date, a substantially growth factor free amniotic membrane referred to herein as GFF-membrane has not been prepared.

Accordingly, in a second aspect, there is provided a substantially growth factor free (GFF) amniotic membrane.

Preferably, the GFF-membrane according to the second aspect is prepared by, or obtainable by, the method according to the first aspect. Preferably, the substantially growth factor free (GFF) amniotic membrane according to the second aspect lacks a spongy layer. Preferably, the substantially growth factor free (GFF) amniotic membrane comprises substantially clinically insignificant soluble growth factors. Preferably, the total concentration of growth factor present in the GFF amniotic membrane is less than 50 ng for a load of 20 μg of total protein extracted from 150 mg of wet tissue, more preferably, below 30 ng, even more preferably, below long, and most preferably, below 5 ng for a load of 20 μg of total protein extracted from 150 mg of wet tissue Preferably, the amniotic membrane is transplant ready.

Using this novel GFF-membrane, which lacks substantially all soluble growth factors, the inventors have developed another novel method for generating a further novel amniotic membrane, referred to herein as an E-membrane, which is enriched with membrane-enriching compounds.

Therefore, according to a third aspect of the present invention, there is provided a method of preparing enriched amniotic membrane (E-membrane), which method comprises contacting substantially growth factor-free (GFF) amniotic membrane with a membrane-enriching compound in conditions suitable to allow uptake of the compound by the GFF amniotic membrane to thereby produce enriched amniotic membrane.

By the term “membrane-enriching compound”, we mean a molecule or chemical capable of conferring a desired beneficial biological effect on the amniotic membrane. The skilled technician will appreciate the various types of membrane-enriching compound with which the amniotic membrane may be enriched. For example, the enrichment compound may comprise a growth factor, for example, EGF, TGF-α, KGF, HGF, bFGF, NGF, TGF-β1, TGF-β2, TGF-β3, TSP-1, PEDF, or any combination thereof.

Alternatively, the membrane-enriching compound may include a steroid; hormone; antimicrobial agent; any other beneficial molecule desired by the surgeon; or any desired compatible combination of the foregoing. Suitable steroids may include Prednisolone phosphate, Prednisolone acetate, Betamethasone, and Dexamethasone. Suitable hormones may include sex steroid hormones, such as oestrogen, progesterone, testosterone. Other membrane-enriching compounds may include biological Antimicrobial peptides, such as Defensins, Cathelicidins, liver-expressed antimicrobial peptides and RNASE 7.

Preferably, the GFF amniotic membrane used in the method according to the third aspect is prepared by, or obtainable by, the method according to the first aspect. Preferably, the contacting step comprises incubating the GFF amniotic membrane in a solution, which solution comprises the desired membrane-enriching compound, under conditions suitable for the compound to be absorbed by the amniotic membrane. This incubation step is also referred to as the installation step. The actual steps in the instillation process may be performed in accordance with any method known per se in the art. The method may comprise contacting the membrane with a combination of membrane enriching compounds, which will be determined by the final use.

The skilled technician will appreciate the required conditions for an effective instillation step. For example, the step may comprise mixing the membrane-enriching compound in a suitable solution, for example, a physiological acceptable buffer such as PBS, and then contacting the GFF amniotic membrane therewith for sufficient time. Preferably, the membrane is washed with or immersed in the solution for sufficient time for absorption to occur. By way of example, the contacting step may be carried out for at least 10 minutes, and preferably at least 20 minutes, at room temperature. It will be appreciated however that the specific conditions required for successful absorption of the compound by the membrane to form the enriched amniotic membrane will be determined by the actual type of compound.

Preferably, the method of the third aspect comprises a further step (following the installation step), which comprises preservation and/or storage of the E-membrane. The preservation step may comprise contacting the GFF amniotic membrane with a preservation compound, for example, dimethyl sulfoxide (DMSO), and preferably, incubating the amniotic membrane with increasing concentrations of DMSO (for example, 4% (v/v), 8% (v/v)), and 12% (v/v)) each for a period of about 5 minutes. The amniotic membrane may then be stored in DMSO (1% v/v) in PBS containing suitable antibiotics, for example gentamicin (160 mg/L) and/or cefiroxime (500 mg/L).

The inventors believe that, to date, an enriched amniotic membrane referred to herein as an E-membrane has not been prepared.

Therefore, in a fourth aspect, there is provided an enriched amniotic membrane (E-membrane) comprising at least one amniotic membrane-enriching compound present at a concentration greater than its corresponding concentration when in normal physiological conditions.

By the term “normal physiological conditions”, we mean the natural biological state of the amniotic membrane when removed from the placenta following child birth.

By the term “enriched”, we mean the amniotic membrane comprises a higher concentration of a membrane-enriching compound as defined herein compared to the concentration of that same compound in amniotic membrane following child birth.

The skilled technician will appreciate how to determine the concentration of compound in the membrane. In some embodiments of the invention, the amniotic membrane may comprise no membrane-enriching compound at all under physiological conditions. Hence, by contacting the GFF membrane with an amniotic membrane enriching compound would result in an enriched amniotic membrane even if only small amounts of the compound are absorbed thereby. Alternatively, the amniotic membrane may have a defined concentration of a compound such as a growth factor under normal physiological conditions, and the GFF amniotic membrane is contacted with the same compound such that upon absorption, the E-membrane comprises a higher level of that growth factor.

The amniotic membrane (i.e. the E-membrane) may comprise at least 10% (w/v), 20% (w/v), 30% (w/v), 40% (w/v), 50% (w/v), 60% (w/v), 70% (w/v), 80% (w/v), 90%, 100% (w/v) higher concentration of a membrane-enriching compound as defined herein compared to the concentration of that same compound in amniotic membrane following child birth. However, it is also envisaged that the E-membrane may comprise 200% (w/v), 3000% (w/v), 4000% (w/v), 500% (w/v) or more, higher concentration of a membrane-enriching compound as defined herein compared to the concentration of that same compound in amniotic membrane following child birth.

Preferably, the enriched membrane of the fourth aspect is prepared by, or obtainable by, the method according to the third aspect.

The inventors believe that the substantially growth factor free (GFF) amniotic membrane according to the second aspect, and also the enriched amniotic membrane according to the fourth aspect will have significant and varied uses in medicine. This is because GFF amniotic membrane is substantially devoid of various growth factors which can, in many circumstances, have a detrimental effect on patients suffering from certain ailments. Hence, use of a growth factor free amniotic membrane would be useful in medicine. In addition, by specifically choosing certain amniotic membrane-enriching compounds in the preparation of the E-membrane, it is possible to tailor design a medically useful amniotic membrane.

Furthermore, the inventors have also realised that the spongy layer of the amniotic membrane that has been removed contains surprisingly high concentrations of various growth factors, such as TGFβ, which is known to promote wound healing that is associated with excessive scarring and also keloid formation. In addition, as shown in FIG. 10, the spongy layer also contains clinically significant concentrations of at least thrombospondin, which is known to be involved with inhibiting angiogenesis (i.e. formation of new blood vessels). Hence, the inventors believe that the spongy layer removed from the amniotic membrane of the method of the first aspect to generate GFF membrane will also have a variety of medical uses due to containing clinically significant concentrations of these various growth factors.

Hence, in a fifth aspect, there is provided substantially growth factor free (GFF) amniotic membrane according to the second aspect, or spongy layer or a component thereof isolated from amniotic membrane, or enriched amniotic membrane (E-membrane) according to the fourth aspect, for use as a medicament.

By the term “component of the spongy layer isolated from amniotic membrane”, we mean biologically functional or active portions of the spongy layer. Hence, it is not necessary to use the entire spongy layer removed from the amniotic membrane. For example, collections of cells may be used therefrom providing they exhibit biological activity in the desired medical treatment or use.

It is preferred that the spongy layer or a component thereof isolated from amniotic membrane is prepared from step (b) of the method according to the first aspect.

The inventors have realised the ability of substantially growth factor free (GFF) amniotic membrane according to the second aspect, or isolated amniotic membrane-derived spongy layer or component thereof, or enriched amniotic membrane (E-membrane) according to the fourth aspect to enhance treatment of wounds or in the treatment of fibrotic disorders. In particular, they believe that they may be used to increase the rate of wound treatment but avoid excessive scarring occurring.

Hence, in a sixth aspect, there is provided use of substantially growth factor free (GFF) amniotic membrane according to the second aspect, or spongy layer or a component thereof isolated from amniotic membrane, or enriched amniotic membrane (E-membrane) according to the fourth aspect, for the manufacture of a medicament for the treatment of wounds, or the treatment of fibrotic disorders.

Preferably, the treatment of the wound results in a prevention or reduction in scarring.

The inventors believe that the substantially growth factor free (GFF) amniotic membrane according to the second aspect, or spongy layer or a component thereof isolated from amniotic membrane, or enriched amniotic membrane (E-membrane) according to the fourth aspect may be used in methods for treating patients.

Hence, in a seventh aspect, there is provided a method of treating a subject suffering from a wound or fibrotic disorder, the method comprising administering to a subject in need of such treatment, a therapeutically effective amount of substantially growth factor free (GFF) amniotic membrane according to the second aspect, or spongy layer or a component thereof isolated from amniotic membrane, or enriched amniotic membrane (E-membrane) according to the fourth aspect.

Transplant ready GFF and E-membranes may be cut to desired size and shape and may be applied surgically to the desired site (for example, on the ocular surface) by use of surgical sutures or tissue adhesive. GFF and E-membranes will be applied to the site as a graft or patch beside, underneath, or on top of the affected area and adjacent healthy tissue as is amply described in the published literature including contributions by the inventors of the present invention. Hence, it should be appreciated that GFF amniotic membrane, or enriched amniotic membrane, or the isolated spongy layer derived from amniotic membrane may be applied directly to the site to be treated. Alternatively, they may be processed into a suitable therapeutically acceptable composition for subsequent application, such as an oil, cream, or liquid, depending on the treatment site.

The inventors believe that the GFF- and E-membranes and also the spongy layer, which has been isolated from amniotic membrane, may be used for preparing a pharmaceutical composition.

Therefore, in an eighth aspect, there is provided a pharmaceutical composition comprising a therapeutically effective amount of substantially growth factor free (GFF) amniotic membrane according to the second aspect, or spongy layer or a component thereof isolated from amniotic membrane, or enriched amniotic membrane (E-membrane) according to the fourth aspect, and a pharmaceutically acceptable diluent, carrier or excipient.

The medicament may he used as surgical material in skin transplantation. In addition, the medicament may be used as a biological dressing for burned skin, skin wounds, and chronic ulcers of the leg; as an adjunctive tissue in surgical reconstruction of artificial vagina, and for repairing omphaloceles; to prevent tissue adhesion in surgical procedures of the abdomen, head, and pelvis. However, the inventors are particularly interested in the use of the medicaments in ophthalmology.

Hence, in a ninth aspect, there is provided use of substantially growth factor free (GFF) amniotic membrane according to the second aspect, or spongy layer or a component thereof isolated from amniotic membrane, or enriched amniotic membrane (E-membrane) according to the fourth aspect, for the manufacture of a medicament for the treatment of ophthalmological conditions.

By way of example, ophthalmological conditions which may be treated include those characterised by a damaged ocular surface. Examples include chromic state of chemical and thermal burns. Ophthalmological conditions which may be treated using GFF according to the second aspect, and/or the E-membrane according to the fourth aspect, include diseases of the eye, for example, Persistent epithelial defects, Nuerotrophic keratitis, Bullous Keratopathy, excision of lesions such as tumour of conjunctiva, and in association with stem cell transplant surgery.

Ophthalmological conditions which may be treated using GFF the spongy layer isolated from the amniotic membrane include acute inflammation, acute state of chemical and thermal burns, and corneal stromal melting diseases, e.g. Rheumatoid Keratopathy, Viral keratitis and bacterial ulcers.

In addition, the inventors of the present invention have accidentally discovered that the physical presence of the spongy layer in situ on the amniotic membrane after surgery can cause major problems. This is because the spongy layer is hygroscopic, and therefore it absorbs water from the ocular surface, which then causes it to swell, pushing the amniotic membrane patch or graft away from the ocular surface, hindering the wound healing process. For this reason, the inventors believe that use of either GFF membrane of the second aspect, or E-membrane of the fourth aspect, both of which lack this hygroscopic spongy layer will have significant medical advantages for treating ophthalmological conditions over and above use of amniotic membranes which include the spongy layer, as in current methodologies.

It will be readily apparent to the skilled person that the ability of the methods and medicaments of the invention to treat wounds, while at the same time reducing scarring mean that these methods and medicaments are of great value in a range of clinical settings. The methods and medicaments of the invention may be used to promote accelerated wound healing with reduced scarring of wounds arising as a result of many different types of injury. For example, the methods and medicaments of the invention may be used in the treatment of penetrating wounds and non-penetrating wounds formed as a result of physical insults or injuries including (but not limited to): grazes, abrasions, surgical incisions, and other surgical procedures (particularly partial thickness grafts of tissues such as the skin), “burns” (which, except for where the context requires otherwise, may be considered to include tissue damage resulting from exposure to either high or low temperature, chemical agents or radiation), and other forms of trauma.

Although the utility of the medicaments and methods of the invention are particularly suited to, and exemplified by, the promotion of accelerated wound healing with reduced scarring in dermal wounds, it will be appreciated that they may also be used to accelerate healing and reduce scarring of wounds in many other tissues. Scars produced by the healing of wounds in tissues other than the skin may also have highly detrimental effects. Specific examples of such tissues include:

(i) Scars occurring as a result of wound healing in the central nervous system. For example, glial scarring can prevent neuronal reconnection (e.g. following neuro-surgery or penetrating injuries of the brain).

(ii) Scars occurring as a result of wound healing in the eye can have many detrimental effects. In the case of wounds of the cornea, scarring can result in abnormal opacity and lead to problems with vision or even blindness. In the case of the retina, scarring can cause retinal detachment or buckling and consequently blindness. Scarring following wound healing in operations to relieve pressure in glaucoma (e.g. glaucoma filtration surgery) frequently results in the failure of the surgery whereby the aqueous humour fails to drain and hence the glaucoma returns.

(iii) Scarring in the heart (e.g. following surgery or myocardial infarction) can give rise to abnormal cardiac function.

(iv) Wound healing involving the abdomen or pelvis often results in adhesion between viscera. For instance, adhesions may form between elements of the gut and the body wall and these can cause twisting in the bowel loop leading to ischaemia, gangrene and the necessity for emergency treatment (if left untreated such conditions may even prove fatal). Likewise, the healing of trauma or incision or incisional wounds in the guts can lead to scarring and scar contracture or strictures which cause occlusion of the lumen of the digestive tract.

(v) Scarring arising as a result of wound healing in the pelvis in the region of the fallopian tubes can lead to infertility.

(vi) Scarring following injury to muscles can result in abnormal contraction and hence poor muscular function.

(vii) Scarring or fibrosis following injury to tendons and ligaments can result in serious loss of function.

Related to the above fact that there are a number of medical conditions known as fibrotic disorders in which excessive fibrosis leads to pathological derangement and malfunctioning of tissue. Fibrotic disorders are characterised by the accumulation of fibrous tissue (predominantly collagens) in an abnormal fashion within the tissue. Accumulation of such fibrous tissues may result from a variety of disease processes. These diseases do not necessarily have to be caused by surgery, traumatic injury or wounding. Fibrotic disorders are usually chronic, and examples include cirrhosis of the liver, liver fibrosis, glomerulonephritis, pulmonary fibrosis, scieroderma, mycocardial fibrosis, fibrosis following myocardial infarction, CNS fibrosis following a stroke, or neuro-degenerative disorders (e.g. Alzheimer's Disease), proliferative vitreoretinopathy (PVR) and arthritis. There is therefore also a need for medicaments which may be used for the treatment of such conditions by regulating (i.e. preventing, inhibiting, or reversing) fibrosis/scarring in these fibrotic disorders.

The ability of the methods and medicaments of the invention to accelerate the healing of wounds is most readily apparent with regard to two properties exhibited by treated wounds. For present purposes a “treated wound” may be considered to be a wound exposed to a therapeutically effective amount of a medicament of the invention, or which has received treatment in accordance with the methods of the invention. Firstly, wounds treated with medicaments in accordance with the invention exhibit an increased rate of epithelialisation as compared to control wounds. The inventors have realised that for normal proliferation and differentiation of corneal epithelial cells, the presence of a normal substrate or basement membrane is essential, facilitating the migration of epithelial cells [26, 27]; reinforcing adhesion of basal epithelial cells [28]; promoting epithelial differentiation [29,30]; preventing epithelial apoptosis [31]. Therefore, a transplanted amniotic membrane in accordance with the invention (i.e. either the GFF amniotic membrane according to the second aspect, or the Enriched amniotic membrane according to the fourth aspect), or the spongy layer isolated from the amniotic membrane, serves as a basement membrane and promotes epithelialisation in the wound site.

Thus the methods and medicaments of the invention promote a more rapid re-constitution of a functional epithelial layer over a wounded area than would otherwise be the case. Secondly, wounds treated with the medicaments of the invention have decreased width compared to control wounds at comparable time points. It will be appreciated that this reduction in wound width ensures that there is a relatively faster rate of wound closure (since there is less width of wound to be closed) and is indicative of the ability of such medicaments to accelerate the healing response.

Accordingly, accelerated wound healing in the context of the present invention should be taken to encompass any increase in the rate of healing of a treated wound as compared with the rate of healing occurring in control-treated or untreated wounds. Preferably, the acceleration of wound healing may be assessed with respect to either comparison of the rate of re-epithelialisation achieved in treated and control wounds, or comparison of the relative width of treated and control wounds at comparable time points. More preferably accelerated wound healing may be defined as comprising both an increased rate of re-epithelialisation and a reduction of wound width compared to control wounds at comparable time points.

Preferably the promotion of accelerated wound healing may give rise to a rate of wound healing that is at least 5%, 10%, 20% or 30% greater than the rate of healing occurring in a control or untreated wound. More preferably the promotion of accelerated wound healing may give rise to a rate of healing that is at least 40%, 50% or 60% greater than healing in a control wound. It is even more preferred that promotion of accelerated wound healing may give rise to a rate of healing that is at least 70%, 80%, or 90% greater than that occurring in control wounds, and most preferably the promotion of accelerated wound healing may give rise to a rate of healing that is at least 100% greater than the rate occurring in control wounds.

There exist a wide range of wound healing disorders that are characterised, or at least partially characterised, by inappropriate failure, delay or retardation of the normal wound healing response. The ability of the methods and medicaments of the invention to promote accelerated wound healing are thus of utility in the prevention or treatment of such disorders. Since the methods and medicaments of the invention are able to bring about the acceleration of wound healing through the promotion of a stimulated re-epithelialisation response (thereby increasing the rate at which the wound closes) it will be appreciated that the methods and medicaments of the invention are particularly advantageous for treatment of wounds of patients that may otherwise be prone to defective, delayed or otherwise impaired re-epithelialisation. For example, it is well known that dermal wounds in the aged exhibit a less-vigorous re-epithelialisation response than do those of younger individuals. There are also many other conditions or disorders in which wound healing is associated with delayed or otherwise impaired re-epithelialisation. For example patients suffering from diabetes, patients with polypharmacy (for example as a result of old age), post-menopausal women, patients susceptible to pressure injuries (for example paraplegics), patients with venous disease, clinically obese patients, patients receiving chemotherapy, patients receiving radiotherapy, patients receiving steroid treatment or immuno-compromised patients may all suffer from wound healing with impaired re-epithelialisation. In many such cases the lack of a proper re-epithelialisation response contributes to the development of infections at the wound site, which may in turn contribute to the formation of chronic wounds such as ulcers. Accordingly it will be appreciated that such patients are particularly likely to benefit from the methods or medicaments of the invention.

Chronic wounds are perhaps the most important example of disorders associated with a delayed wound healing response. A wound may be defined as chronic if it does not show any healing tendency within eight weeks of formation when subject to appropriate (conventional) therapeutic treatment. Well-known examples of chronic wounds include venous ulcers, diabetic ulcers and decubitus ulcers, however chronic wounds may arise from otherwise normal acute injuries at any time. Typically chronic wounds may arise as a result of infection of the wound site, inadequate wound treatment, or as a sequitur of progressive tissue breakdown caused by venous, arterial, or metabolic vascular disease, pressure, radiation damage, or tumour.

It will be appreciated that the methods and medicaments of the invention may be utilised in the treatment of existing chronic wounds in order to promote their healing. The methods and medicaments may promote the re-epithelialisation of chronic wounds, thereby bringing about healing and closure of the disorder, while also reducing scarring associated with wound healing. The prevention of scarring in such contexts may be particularly advantageous since chronic wounds may typically extend over relatively large portions of a patient's body.

In addition, or alternatively, to their use in the treatment of existing chronic wounds, the methods and medicaments of the invention may be used to prevent acute wounds of patients predisposed to impaired wound healing developing into chronic wounds. Since the methods and medicaments of the invention promote epithelial coverage of the damaged site they are able to reduce the likelihood of a treated wound becoming infected. Similarly, this promotion of re-epithelialisation may be of benefit in the treatment of chronic wounds arising as a result of other conditions such as diabetes or venous disease.

The ability of medicaments in accordance with the invention to promote accelerated wound healing, preferably with reduced scarring, without impairing the naturally occurring inflammatory response provides a marked advantage in that the cells involved in the inflammatory response (and more particularly factors released or secreted by such cells) play a major role in controlling the normal progression of the healing response, thereby bringing about wound closure and repair. Thus the medicaments and methods of the invention are of particular benefit in the promotion of accelerated wound healing with reduced scarring in patients predisposed to deficient wound healing since the methods and medicaments do not bring about the adverse effects that may be associated with reduced inflammatory activity.

A further group of patients that may derive particular benefit from the methods and medicaments of the invention are those in which the immune system is compromised (for example patient undergoing chemotherapy or radiotherapy, or those suffering from HIV infection). It is well recognised that wounds of immuno-compromised patients, who may be unable to mount a normal inflammatory response after wounding, tend to be associated with poor healing outcomes. These effects may be caused both by the absence of growth factors and other products released by inflammatory cells, and also the increased risk of wound infection with may contribute to prolonged and defective healing. Accordingly, in a preferred embodiment of the invention the medicaments of the invention may be used to prevent or reduce scarring in contexts where it is preferred to maintain the naturally occurring inflammatory response.

The ability of medicaments and methods of the invention to promote accelerated wound healing with reduced scarring (and without anti-inflammatory activity) is also of use in more general clinical contexts. Examples of these further benefits may be considered with reference to the healing of wounds by primary, secondary or tertiary intention, as described below.

For the purposes of the present invention healing by primary intention may be considered to involve the closure by surgical means (such as sutures, adhesive strips or staples) of opposing edges of a wound. Healing by primary intention is typically employed in the treatment of surgical incisions or other clean wounds, and is associated with minimal levels of tissue loss. The skilled person will recognise that since medicaments or methods in accordance with the invention are capable of reducing wound width they facilitate the joining of opposing wound edges, and thus may be beneficial in wound healing by primary intention. Furthermore, since the methods and medicaments reduce wound width but do not disrupt the normal inflammatory response they are able to promote accelerated wound healing with reduced scarring without increasing the risk of infection.

For the purposes of the present invention healing by secondary intention may be considered to constitute the closure of wounds by the wound healing process, without direct surgical intervention. Wounds to be healed by secondary intention may be subject to continued care (for example the dressing and re-dressing of the wound as well as the application of suitable medicaments), but it is the natural processes of granulation tissue formation and re-epithelialisation that bring about the closure of the wound. It will be appreciated that since medicaments and methods of the invention are able to increase the rate of re-epithelialisation as compared to that occurring in control wounds they have utility in the promotion of wound healing by secondary intention.

Furthermore, since the methods and medicaments of the invention do not reduce the inflammatory response at the injured site (which response constitutes a vital mediator of granulation tissue formation), they are not associated with the retardation of healing by secondary intention that may occur as a result of the use of agents having anti-inflammatory activity. That methods and medicaments of the invention do not inhibit granulation tissue formation is illustrated by the highly comparable degrees of cellularity exhibited by treated and control wounds.

Healing by tertiary intention may be considered to comprise the surgical closure of a wound that has previously been left open to allow at least partial granulation tissue formation and re-epithelialisation. The properties of the methods and medicaments of the invention that make them suitable for use in healing by primary or secondary intention are also beneficial in the context of promoting wound healing by tertiary intention.

It is known that TGFβ promotes scarring during wound healing. Accordingly, because GFF amniotic membrane, and Enriched amniotic membrane are devoid of this growth factor, scarring during wound healing can be avoided. This is particularly important when treating ophthalmological conditions as a scar on the eye will often result in loss of vision quality. The prevention or reduction of scarring within the context of the present invention should be understood to encompass any reduction in scarring as compared to the level of scarring occurring in a control-treated or untreated wound. Although medicaments of the invention may be used to promote accelerated wound healing with reduced scarring in the wide range of tissues described above, it is preferred that they be used to accelerate healing and reduce scarring of the skin.

The reduction of dermal scarring achieved using methods and medicaments of the invention may be assessed with reference to either the microscopic or, preferably, macroscopic appearance of a treated scar as compared to the appearance of an untreated scar. More preferably the reduction in scarring may be assessed with reference to both macroscopic and microscopic appearance of a treated scar. For the present purposes a “treated scar” may be defined as a scar formed on healing of a treated wound, whereas an “untreated scar” may be defined as the scar formed on healing of an untreated wound, or a wound treated with placebo or standard care. Suitable comparison scars may preferably be matched to the treated scar with reference to scar age, site, size and patient.

In considering the macroscopic appearance of a scar resulting from a treated wound, the extent of scarring, and hence the magnitude of any reduction in scarring achieved, may be assessed with reference to any of a number of parameters. Suitable parameters for the macroscopic assessment of scars may include: colour of the scar; height of the scar; surface texture of the scar; and the stiffness of the scar.

A treated scar will preferably demonstrate a reduction in scarring as assessed with reference to at least one of the parameters for macroscopic assessment set out above. More preferably a treated scar may demonstrate reduced scarring with reference to at least two of the parameters, even more preferably at least three of the parameters, and most preferably all four of these parameters. An overall assessment of scarring may be made using, for example, a Visual Analogue Scale or a digital assessment scale. Suitable parameters for the microscopic assessment of scars may include: Thickness of extracellular matrix (ECM) fibres; orientation of ECM fibres; ECM composition of the scar; and the cellularity of the scar.

A treated scar will preferably demonstrate a reduction in scarring as assessed with reference to at least one of the parameters for microscopic assessment set out above. More preferably a treated scar may demonstrate reduced scarring with reference to at least two of the parameters, even more preferably at least three of the parameters, and most preferably all four of these parameters. A reduction or an improvement in scarring of a treated wound may farther be assessed with reference to suitable parameters used in the:
i) macroscopic clinical assessment of scars, particularly the assessment of scars upon a subject;
ii) assessment of photographic images of scars; and
iii) microscopic assessment of scars, for example by histological analysis of the microscopic structure of scars.

It will be appreciated that an improvement in scarring of a treated wound may be indicated by improvement of one or more such suitable parameters, and that in the case of an improvement as assessed with reference to a number of parameters that these parameters may be combined from different assessment schemes (e.g. improvement in at least one parameter used in macroscopic assessment and at least one parameter used in microscopic assessment). A reduction or improvement in scarring may be demonstrated by an improvement in one or more parameters indicating that a treated scar more closely approximates unscarred skin with reference to the selected parameter(s) than does an untreated or control scar.

Suitable parameters for the clinical measurement and assessment of scars may be selected based upon a variety of measures or assessments including those described by Beausang et al (1998) and van Zuijlen et al (2002). Typically, suitable parameters may include: Assessment with regard to Visual Analogue Scale (VAS) scar score; Scar height, scar width, scar perimeter, scar area or scar volume; Appearance and/or colour of scar compared to surrounding unscarred skin; Scar distortion and mechanical performance; Scar contour and scar texture.

The inventors have found that, since the methods and medicaments of the invention are able to promote re-epithelialisation, they are particularly effective in the treatment of all injuries involving damage to an epithelial layer. Such injuries are exemplified by, but not limited to, injuries to the skin, in which the epidermis is damaged. It will however be appreciated that the methods and medicaments of the invention are also applicable to other types of wounds in which epithelia are damaged, such as injuries involving the respiratory epithelia, digestive epithelia or epithelia surrounding internal tissues or organs (such as the epithelia of the peritoneum).

The healing of wounds involving the peritoneum (the epithelial covering of the internal organs, and/or the interior of the body cavity) may frequently give rise to adhesions. Such adhesions are a common sequitur of surgery involving gynaecological or intestinal tissues. The inventors believe that the ability of the methods and medicaments of the invention to accelerate the regeneration of the peritoneum while reducing scarring may reduce the incidence of inappropriate attachment of portions of the peritoneum to one another, and thereby reduce the occurrence of adhesions. Accordingly, the use of the methods and medicaments of the invention to prevent the formation of intestinal or gynaecological adhesions represents a preferred embodiment of the invention. Indeed the use of the methods or medicaments of the invention in the healing of any wounds involving the peritoneum is a preferred embodiment.

The use of the methods and medicaments of the invention to stimulate re-epithelialisation (and thus promote accelerated wound healing) while reducing scarring is also particularly effective in the treatment of wounds associated with grafting procedures. Treatment using the methods and medicaments of the invention is of benefit both at a graft donor site (where it can aid the re-establishment of a functional epithelial layer while reducing scar formation), and also at graft recipient sites (where the anti-scarring effects of the treatment reduce scar formation, while the accelerated healing promotes integration of the grafted tissue). The inventors have found that the methods and medicaments of the invention confer advantages in the contexts of grafts utilising skin, artificial skin, or skin substitutes.

The inventors have found that the methods and medicaments of the invention are able to promote accelerated wound healing with reduced scarring when administered either prior to wounding, or once a wound has already been formed. The methods or medicaments of the invention may be used prophylactically, at sites where no wound exists but where a wound that would otherwise give rise to a scar or chronic wound is to be formed. By way of example medicaments in accordance with the invention may be administered to sites that are to undergo wounding as a result of elective procedures (such as surgery), or to sites that are believed to be at elevated risk of wounding. It may be preferred that the medicaments of the invention are administered to the site immediately prior to the forming of a wound (for example in the period up to six hours before wounding) or the medicaments may be administered at an earlier time before wounding (for example up to 48 hours before a wound is formed). The skilled person will appreciate that the most preferred times of administration prior to formation of a wound will be determined with reference to a number of factors, including the formulation and route of administration of the selected medicament, the dosage of the medicament to be administered, the size and nature of the wound to be formed, and the biological status of the patient (which may determined with reference to factors such as the patient's age, health, and predisposition to healing complications or adverse scarring). The prophylactic use of methods and medicaments in accordance with the invention is a preferred embodiment of the invention, and is particularly preferred in the promotion of accelerated wound healing with reduced scarring in the context of surgical wounds.

The methods and medicaments of the invention are also able to promote accelerated wound healing if administered after a wound has been formed. It is preferred that such administration should occur as early as possible after formation of the wound, but agents of the invention are able to promote accelerated wound healing with reduced scarring at any time up until the healing process has been completed (i.e. even in the event that a wound has already partially healed the methods and medicaments of the invention may be used to promote accelerated wound healing with reduced scarring in respect of the remaining un-healed portion). It will be appreciated that the “window” in which the methods and medicaments of the invention may be used to promote accelerated wound healing with reduced scarring is dependent on the nature of the wound in question (including the degree of damage that has occurred, and the size of the wounded area). Thus in the case of a large wound the methods and medicaments of the invention may be administered relatively late in the healing response yet still be able to promote accelerated wound healing with reduced scarring. The methods and medicaments of the invention may, for instance, preferably be administered within the first 24 hours after a wound is formed, but may still promote accelerated wound healing with reduced scarring if administered up to ten, or more, days after wounding.

The methods and medicaments of the invention may be administered on one or more occasions as necessary in order to promote accelerated wound healing with reduced scarring. For instance therapeutically effective amounts of the medicaments may be administered to a wound as often as required until the healing process has been completed. By way of example, the medicaments of the invention may be administered daily or twice daily to a wound for at least the first three days following the formation of the wound.

Most preferably the methods or medicaments of the invention may be administered both before and after formation of a wound. It will be appreciated that the amount of a medicament of the invention that should be applied to a wound depends on a number of factors such as the biological activity and bioavailability of the agent present in the medicament, which in turn depends, among other factors, on the nature of the agent and the mode of administration of the medicament.

Generally when medicaments in accordance with the invention are used to treat existing wounds the medicament should be administered as soon as the wound has occurred (or in the case of wounds that are not immediately apparent, such as those at internal body sites, as soon as the wound has been diagnosed). Therapy with methods or medicaments in accordance with the invention should continue until the healing process has been accelerated, and scarring reduced, to a clinician's satisfaction.

Frequency of administration will depend upon the biological half-life of the medicament used. Typically a cream or ointment containing an agent of the invention should be administered to a target tissue such that the concentration of the amniotic membrane or spongy layer derived therefrom at a wound is maintained at a level suitable for having a therapeutic effect. This may require administration daily or even several times daily.

Medicaments of the invention, may be administered by any suitable route capable of achieving the desired effect of promoting wound healing with reduced scarring, but it is preferred that the medicaments be administered locally at the wound site. The inventors believe that promotion of accelerated wound healing with reduced scarring may be effected by the administration of an agent of the invention by injection at the wound site. For instance, in the case of dermal wounds, agents of the invention may be administered by means of intradermal injection. Thus a preferred medicament in accordance with the invention comprises an injectable solution of an agent of the invention (e.g. for injection around the margins of a site of epithelial damage or a site likely to be damaged). Suitable formulations for use in this embodiment of the invention are considered below.

Alternatively, or additionally, medicaments of the invention may also be administered in a topical form to promote accelerated wound healing with reduced scarring. Such administration may be effected as part of the initial and/or follow up care for the wounded area. The inventors believe that the promotion of accelerated wound healing is particularly improved by topical application of an agent of the invention to a wound (or, in the case of prophylactic application, to a tissue or site where a wound is to be formed).

Compositions or medicaments containing GFF amniotic membrane of the second aspect, E-amniotic membrane of the fourth aspect, or isolated spongy layer derived from amniotic membrane may take a number of different forms depending, in particular on the manner in which they are to be used. Thus, for example, they may be in the form of a liquid, ointment, cream, gel, hydrogel, powder or aerosol. All of such compositions are suitable for topical application to a wound, which is a preferred means of administering GFF amniotic membrane of the second aspect, E-amniotic membrane of the fourth aspect, or isolated spongy layer derived from amniotic membrane, to a subject (e.g. a person or animal) in need of treatment.

The GFF amniotic membrane of the second aspect, E-amniotic membrane of the fourth aspect, or isolated spongy layer derived from amniotic membrane may be provided on a sterile dressing or patch, which may be used to cover a site of epithelial damage to be treated. It will be appreciated that the vehicle of the composition comprising agents of the invention should be one that is well tolerated by the patient and allows release of the active agent to the wound. Such a vehicle is preferably biodegradeable, bioresolveable, bioresorbable and/or non-inflammatory.

Compositions comprising GFF amniotic membrane of the second aspect, E-amniotic membrane of the fourth aspect, or isolated spongy layer derived from amniotic membrane may be used in a number of ways. Thus, for example, a composition may be applied in and/or around a wound in order to promote accelerated wound healing with reduced scarring. If the composition is to be applied to an “existing” wound, then the pharmaceutically acceptable vehicle will be one which is relatively “mild” i.e. a vehicle which is biocompatible, biodegradable, bioresolvable and non-inflammatory.

An amniotic membrane (GFF- or E-membrane) or spongy layer derived from amniotic membrane may be incorporated within a slow or delayed release device. Such devices may, for example, be placed on or inserted under the skin and amniotic membrane (GFF- or E-membrane) of spongy layer derived from amniotic membrane may be released over days, weeks or even months. Such a device may be particularly useful for patients (such as those suffering from chronic wounds) that require long-term promotion of accelerated wound healing with reduced scarring. The devices may be particularly advantageous when used for the administration of amniotic membrane (GFF- or E-membrane) or spongy layer derived from amniotic membrane, which would normally require frequent administration (e.g. at least daily administration by other routes).

Daily doses of amniotic membrane (GFF- or E-membrane) or spongy layer derived from amniotic membrane may be given as a single administration (e.g. a daily application of a topical formulation or a daily injection). Alternatively, the amniotic membrane (GFF- or E-membrane) or spongy layer derived from amniotic membrane may require administration twice or more times during a day. In a further alternative, a slow release device may be used to provide optimal doses of amniotic membrane (GFF- or E-membrane) or spongy layer derived from amniotic membrane to a patient without the need to administer repeated doses.

In one embodiment a pharmaceutical vehicle for administration of amniotic membrane (GFF- or E-membrane) or spongy layer derived from amniotic membrane may be a liquid and a suitable pharmaceutical composition would be in the form of a solution. In another embodiment, the pharmaceutically acceptable vehicle is a solid and a suitable composition is in the form of a powder or tablet. In a further embodiment the agent of the invention may be formulated as a part of a pharmaceutically acceptable transdermal patch.

A solid vehicle can include one or more substances which may also act as flavouring agents, lubricants, solubilizers, suspending agents, fillers, glidants, compression aids, binders or tablet-disintegrating agents; it can also be an encapsulating material. In powders, the vehicle is a finely divided solid which is in admixture with the finely divided amniotic membrane (GFF- or E-membrane) or spongy layer derived from amniotic membrane. In tablets, the amniotic membrane (GFF- or E-membrane) or spongy layer derived from amniotic membrane is mixed with a vehicle having the necessary compression properties in suitable proportions and compacted in the shape and size desired. The powders and tablets preferably contain up to 99% of the amniotic membrane (GFF- or E-membrane) or spongy layer derived from amniotic membrane. Suitable solid vehicles include, for example, calcium phosphate, magnesium stearate, talc, sugars, lactose, dextrin, starch, gelatin, cellulose, polyvinylpyrrolidine, low melting waxes and ion exchange resins.

Liquid vehicles may be used in preparing solutions, suspensions, emulsions, syrups, elixirs and pressurized compositions. The amniotic membrane (GFF- or E-membrane) or spongy layer derived from amniotic membrane can be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilizers, emulsifiers, buffers, preservatives, sweeteners, flavouring agents, suspending agents, thickening agents, colors, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be halogenated hydrocarbon or other pharmaceutically acceptable propellant.

Liquid pharmaceutical compositions which are sterile solutions or suspensions can be utilized by for example, intramuscular, intrathecal, epidural, intraperitoneal, intradermal or subcutaneous injection. Sterile solutions can also be administered intravenously. The amniotic membrane (GFF- or E-membrane) or spongy layer derived from amniotic membrane may be prepared as a sterile solid composition which may be dissolved or suspended at the time of administration using sterile water, saline, or other appropriate sterile injectable medium. Vehicles are intended to include necessary and inert binders, suspending agents, lubricants and preservatives.

Compositions of amniotic membrane (GFF- or E-membrane) or spongy layer derived from amniotic membrane are suitable to be used for promoting accelerated wound healing with reduced scarring in the cornea. Corneal wounds may result from trauma to the eye arising as a result of accidental injury (as considered above) or as a result of surgical operations (e.g. laser surgery on the cornea). In this case a preferred medicament of the invention may be in the form of an eye drop.

Amniotic membrane (GFF- or E-membrane) or spongy layer derived from amniotic membrane may be used in a range of “internal” wounds (i.e. wounds occurring within the body, rather than on an external surface). Thus for example, medicaments in accordance with the invention may be formulated for inhalation for use in wounds arising in the lungs or other respiratory epithelia.

Known procedures, such as those conventionally employed by the pharmaceutical industry (e.g. in vivo experimentation, clinical trials etc), may be used to establish specific formulations of compositions comprising amniotic membrane (GFF- or E-membrane) or spongy layer derived from amniotic membrane and precise therapeutic regimes for administration of such compositions (such as daily doses of the active agent and the frequency of administration).

A suitable daily dose of an amniotic membrane (GFF- or E-membrane) or spongy layer derived from amniotic membrane able to promote accelerated wound healing with reduced scarring depends upon a range of factors including (but not limited to) the nature of the tissue wounded, area and/or depth of the wound to be treated, the severity of the wound, and the presence or absence of factors predisposing to pathological scar or chronic wound formation. Typically the amount of an amniotic membrane (GFF- or E-membrane) or spongy layer derived from amniotic membrane required for the treatment of sites of epithelial damage will be within the range of 0.001 ng to 100 mg of the agent per 24 hours, although this figure may be modified upwards or downwards in response to the factors outlined above. The amount of the amniotic membrane (GFF- or E-membrane) or spongy layer derived from amniotic membrane to be administered may preferably be 50 to 500 ng per linear centimetre of epithelial damage.

Effective medicaments may suitably comprise amniotic membrane (GFF- or E-membrane) or spongy layer derived from amniotic membrane concentrations of between 1 ng per 100 μl medicament and 10 μg per 100 μl medicament. The optimal concentration of amniotic membrane (GFF- or E-membrane) or spongy layer derived from amniotic membrane to be used in a particular medicament will be determined by a range of factors, including the nature of the medicament, the route of administration, and the tissue in which wound healing is to be promoted. The ways in which preferred concentrations may be calculated based on such factors are conventional, and will be well known to those skilled in the art.

The inventors believe that medicaments able to promote accelerated wound healing with reduced scarring may comprise amniotic membrane (GFF- or E-membrane) or spongy layer derived from amniotic membrane at concentrations of as little as 1, 10, 25, 125 or 250 ng peptide per 100 μl medicament.

Amniotic membrane (GFF- or E-membrane) or spongy layer derived from amniotic membrane may be used to promote accelerated wound healing with reduced scarring as a monotherapy (e.g. through use of medicaments of the invention alone). Alternatively the methods or medicaments of the invention may be used in combination with other compounds or treatments for the promotion of wound healing. Suitable treatments that may be used as parts of such combination therapies will be well known to those skilled in the art.

In a further aspect, the invention provides a method of processing amniotic membranes to remove the ‘spongy’ growth factor rich layer and thereby generate a GFF-membrane, which method comprises steps of:
(a) Isolating the amniochorionic membrane from the placenta by cutting around the periphery of the placental body
(b) Washing the amniochorion thoroughly in sterile solution (preferably saline (0.9% (w/v) NaCl) to remove any excess blood
(c) Separating the amniotic membrane from the chorion
(d) Further soaking of the amniotic membrane in excess chilled sterile solution (preferably saline)
(e) Removal of only the growth factor rich mucinous spongy layer from the amniotic membrane
(f) Preservation and/or Storage of the GFF-membrane or further processing to generate an E-membrane.

In a still further aspect the invention provides a method of instilling molecules and/or compounds into the GFF-membrane to generate an E-membrane, which method comprises steps of:
(a) post-storage or post-preservation preparation of the GFF-membrane for GFF-membranes that have been stored and/or preserved
(b) incubating the GFF-membrane (pre or post preservation and/or storage) in a solution containing the desired molecule/compound or combination of molecules/compounds
(c) post-incubation processing of the E-membrane either in preparation for preservation and/or storage or in preparation for use.

In a further aspect, the invention also provides GFF-membrane and E-membrane for use in clinical and surgical techniques.

In a further aspect, the invention provides a method of isolating and retaining the factor rich ‘spongy layer’, which will be processed and modified to generate a formulated substance of known factor content, which method comprises:
a) Collection of the spongy layer and the solution (chilled sterile solution preferably saline) used in its separation from the amniotic membrane during processing;
b) Concentration of the above (a) two components;
c) Adaptation of the gelatinous substance into a manageable format;
d) Quantitation of the beneficial growth factor or factors desired;
e) Final dilution/further concentration to a pre-determined quantity;
f) Preservation/storage of dispensation as drops/solution or ointment for clinical use.

It is a further object of the present invention to formulate the spongy layer thus separated, into drops, ointment or solution for the treatment of wounds.

All of the features described herein (including any accompanying claims, abstract and drawings), and/or all of the steps of any method or process so disclosed, may be combined with any of the above aspects in any combination, except combinations where at least some of such features and/or steps are mutually exclusive.

The present invention will be further understood with reference to the following Examples, and the accompanying Figures in which:

FIG. 1 shows a diagrammatic representation of the foetal membrane, taken from Bourne et al 1960 [43], showing the five layers of the amniotic membrane; (Innermost first) Amniotic epithelium, Basement membrane, Compact layer, fibroblast layer and spongy layer. The subsequent four layers; Cellular layer, reticular layer, Pseudo-basement membrane and the trophoblast layer form the underlying chorion;

FIG. 2 shows gel electrophoretic protein visualisation in amniotic membranes. Levels of TGF-β1 protein expression in AM. Crude protein extracts from fresh AM (1-4), processed AM (5-8), and spongy layer (9-11) were separated on denaturing PAGE mini-gels under reducing conditions, and Coomassie stained to determine equal load (A), or western blotted to PVDF, and detected with anti-TGF-β1 antibody (B). Four AMs were used i) 1,5,9; ii) 2,6,10; iii), 3,7,11; and iv), 4,8. A representative experiment out of five performed is shown;

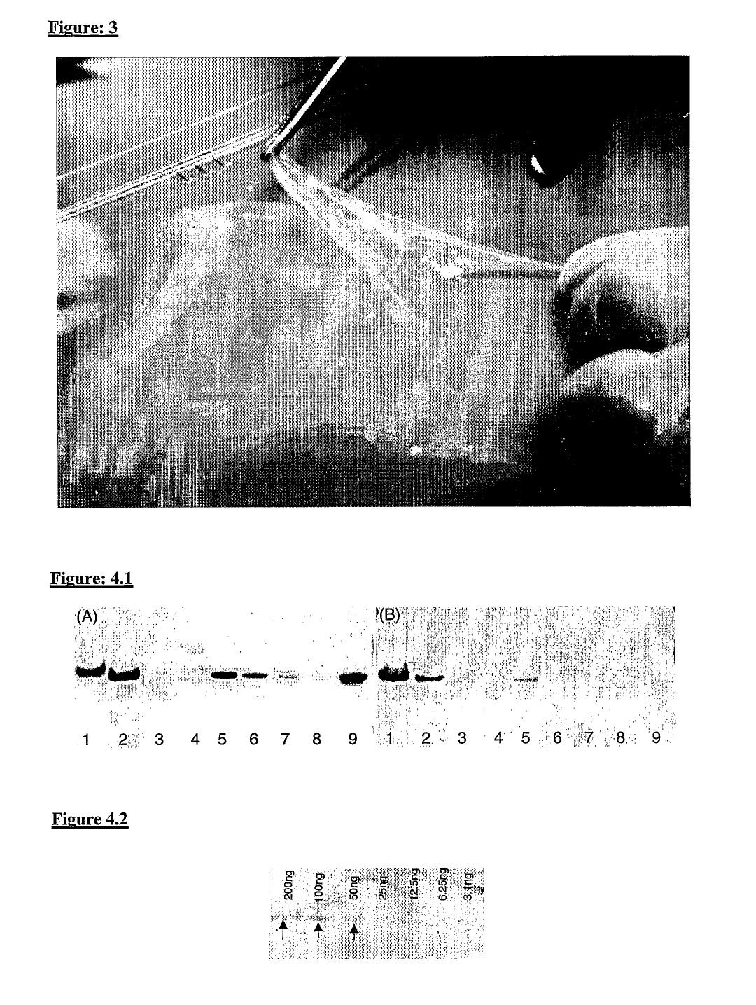

FIG. 3 shows spongy layer removal from TRAM using the modified procedure. Spongy layer removal from TRAM using the modified procedure. Forceps are used to pull the spongy layer away from the AM. A brief wash in fresh saline was performed to remove any loose debris. Removing the spongy layer from the AM removed any remaining contaminating blood from the amnion leaving a white and translucent membrane;

FIG. 4.1 shows immunodetection of TGF-β1 in amniotic membranes. Levels of TGF-β1 protein expression in AM, removed during processing. Total protein was assayed and equal amounts of 20 μg were loaded in each lane. Proteins were separated on denaturing PAGE mini-gels under reducing conditions; western blotted to PVDF and detected with anti-TGF-β1 antibody. Lanes shown are Spongy layer (1), Chorion (2), Fresh AM (3), Processed AM (4), 10× Storage medium (5), 10× Wash 1 (6), 10× Wash 2 (7), 10× Wash 3 (8), and 10× pooled washes (9). Representative experiments (A and B) out of fifteen performed are shown;

FIG. 4.2 shows titration of human TGF-β1 purified form platelets to establish the levels of TGF-β1 in AM and spongy layer. TGF-β1 purified from platelets (RnD, UK) was titrated from 200 ng down to 3.1 ng and the respective amounts were loaded in each well. Proteins were separated on denaturing PAGE mini-gels under reducing conditions; western blotted to PVDF and detected with anti-TGF-β1 antibody. Lanes shown are 200 ng, 100 ng, 50 ng, 12.5 ng, 6.25 ng, and 3.1 ng. Positive staining is indicated by the arrows, and was down to 50 ng. The staining intensity of the spongy layer (FIG. 4.1) was at least 4 fold greater than that observed for 200 ng human TGF-β1 load;

FIG. 5 shows levels of TGF-β1 protein expression in AM and spongy layer, not removed during processing. Levels of TGF-β1 protein expression in AM, not removed during processing. Total protein was assayed and equal amounts of 20 μg were loaded in each lane. Proteins were separated on denaturing PAGE mini-gels under reducing conditions; western blotted to nitrocellulose and reacted with anti-TGF-β1 antibody. Lanes shown are Spongy layer (1), Fresh AM (2), Processed AM (3), 10× Storage medium (4), 10× Wash 1 (5), 10× Wash 2 (6), 10× Wash 3 (7), and 10× pooled washes (8). A representative experiment out of seven performed is shown;

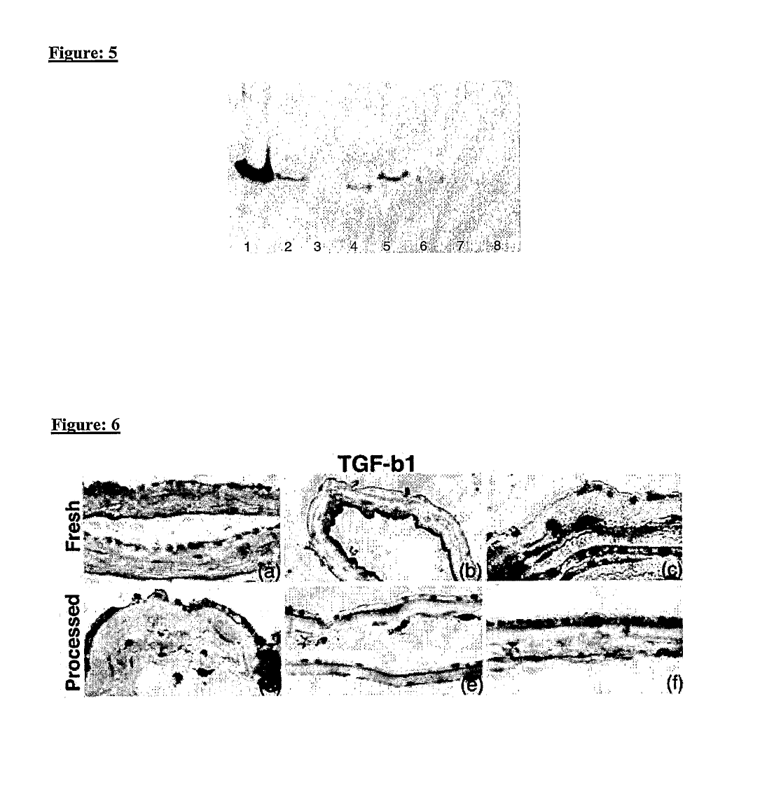

FIG. 6 shows immunohistochemiststry for TGF-β1 in amniotic membrane obtained fresh (a-c) and corresponding preserved processed membrane (d-f), from three different membranes. Images shown are 400× magnification;

FIG. 7 shows a PDQuest image composite demonstrating intra-sample reproducibility. 2D electrophoresis gels generated from TRAM. Comparable zoomed areas of two replicate gels from eight membrane samples are shown; sample 1(A,B), sample (C,D), sample 3 (E,F), sample 4 (G,H), sample 5 (I,J), sample 6 (K,L), sample 7 (M,N), sample 8 (O,P);

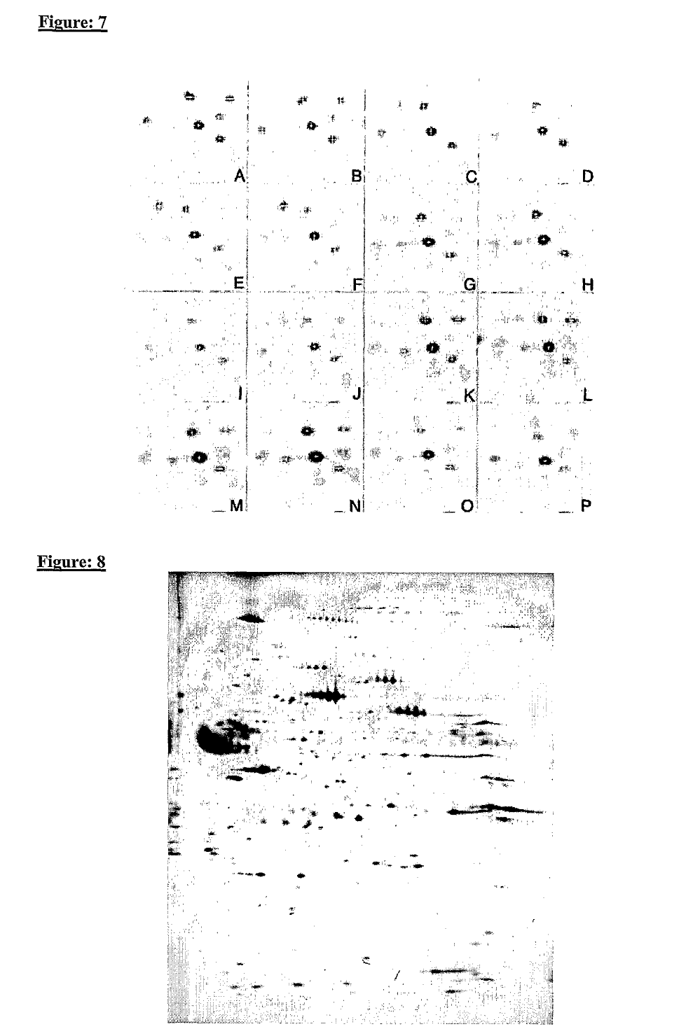

FIG. 8 shows 2D electrophoresis of spongy layer proteins;

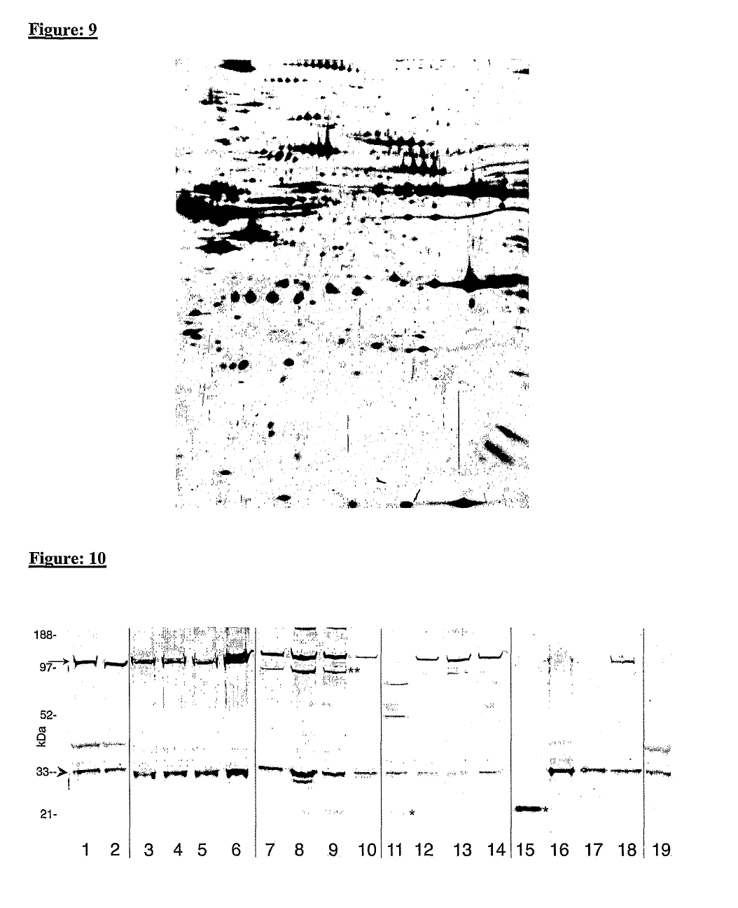

FIG. 9 shows a 2D electrophoretic composite overlay of TRAM and spongy layer. 2 DE gel from TRAM matched with spongy layer. The blue indicates proteins detected only in the TRAM, the red indicates proteins detected only in the spongy layer. Spots detected in both are cancelled out and appear black;

FIG. 10 shows immunohistochemistry for thrombospondin in AM and spongy layer. TSP-1 detection in AM. Total protein was assayed and 20 μg was loaded in each lane. Proteins were separated on denaturing PAGE mini-gels under reducing conditions, western blotted to PVDF and TSP-1 detected with anti-TSP antibody cocktail (AB-11, Neomarkers). Proteins were extracted from fresh AM (lanes 1,2), TRAM (lanes 3 to 6), storage medium (lanes 7 to 10), pooled wash media (lanes 11 to 14), spongy layer removed during processing (lanes 15 to 18), and chorion (lane 19). Samples shown correspond to four membranes; AM1 (lanes 1,5,9,13,17,18), AM2 (lanes 2,6,10,14,19), AM3 (lanes 3,7,11,1) and AM4 (lanes 4,8,12,16). 127 kDa TSP-1 parent protein (Arrow), MS identified fragment (arrow head), large fragment (**) and small fragment (*) are indicated. Size markers (Multimark, Invitrogen) are indicated. A representative experiment out of three performed is shown; and

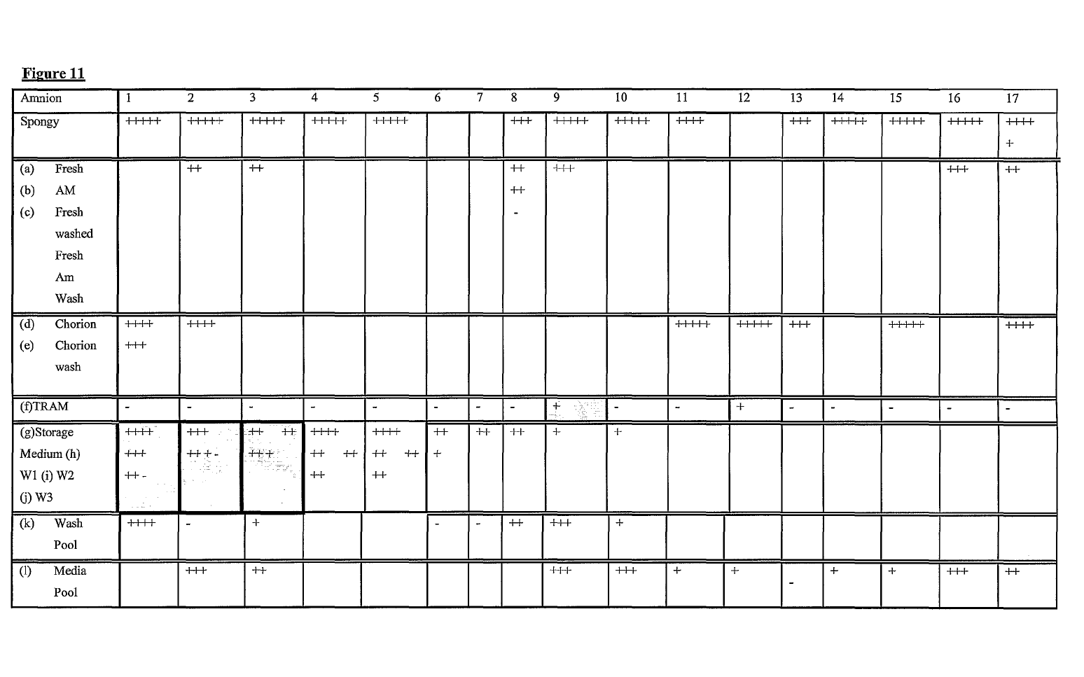

FIG. 11 is a table showing TGF-β1 staining intensities in various AM fractions obtained during processing and preparation. Intensity graded from most intense (+++++) to least intense (+), and not detected (−). Blank indicates no sample was screened. Protein was extracted from samples collected at various stages from seventeen foetal membranes processed, preserved and then prepared as for transplantation (1-17). Samples were from fresh AM (a) and chorion (d); AM prepared for preservation and then washed in storage medium (b), and the retained concentrated wash (c); media used to wash chorion, after preservation (e); concentrated AM storage medium (g); concentrated individual sequential washes from AM after preservation (h-j); concentrated pooled AM washes, excluding storage medium (k); concentrated pooled storage and wash media (l); TRAM (f). Staining was representative of a quantitated 20 μg total protein load per sample. Examples of sequential elution (boxed), prolonged elution (underlined), and processed membranes staining positive for TGF-β1 (shaded) are indicated. Representative experiment (table) out of three performed is shown.

### EXAMPLES

### Example 1

### Demonstrating the Presence of a Growth Factor (for Instance TGF-β1) in AM

As an example of a growth factor, the inventors have evaluated the presence and location of TGF-β1 in AM pre and post processing and preservation (namely fresh AM and transplant ready AM (TRAM)). TGF-β1 is the prototypical member of the TGF-β superfamily; members of the TGF-β superfamily have very diverse and profound effects on various stages of development as well as maintaining tissue function and integrity during adult life[39]. TGF-β is known to regulate proliferation and differentiation of cells, inflammation, wound healing, angiogenesis, ECM remodelling in a variety of tissues and organs, and embryonic development. Almost all cells in the body produce TGFβ and have receptors for it. TGF-β is reported to be instrumental in stimulating fibroblasts and has been implicated as the key mediator of fibrogenesis in various tissues[40]. During wound healing, TGF-β increases matrix protein synthesis and decreases matrix protein degradation, resulting in tissue fibrosis and scarring.

### Amniotic Membrane Procurement

AMs eligible for use in transplantation must be obtained via an elective caesarean. This guarantees the integrity of the membrane is maintained, reducing the risk of contamination, and ensuring sterility. Prospective donors were identified in the pre-clerking clinic, with help of the attending midwives, 2-3 days prior to the elective caesarean. Individuals with a poor social history such as drugs misuse alcohol abuse and multiple sexual partners were classified as incompatible for donor eligibility and were therefore excluded. Informed consent was obtained from all donors, according to an approved ethics procedure, and copies of the information sheet and consent forms were kept in the donor's medical records. Screening of communicable diseases, specifically syphilis, human immuno-deficiency virus (HIV) and hepatitis was carried out, firstly as mandatory in the third trimester, as close to the date of the caesarean as possible, followed by a repeat, six months post delivery. Only when both test were negative, was the tissue be used for surgery. Collection of the AM was within 15 minutes post delivery, maintaining sterile conditions at all times. The membrane was processed and prepared for storage immediately.

### Standard Method for Amniotic Membrane Processing and Storage

Processing and preparation of the AM was performed under sterile conditions, in a class II lamina-flow cabinet. The method was a modified protocol taken from Tsuboto et al[41]. The amniochorionic membrane was isolated from the placenta by cutting around the periphery of the placental body. Amniochorion was washed thoroughly in sterile saline (0.9% (w/v) NaCl) to remove any excess blood followed by separation of the AM from the chorion. Starting from an outer edge of the amniotic reflectum, the membranes were pulled apart, initially using blunt forceps, until a AM flap developed and then by hand, pulling the membranes apart in opposite directions. Separation of the membranes exposed the spongy layer, to which excess blood would associate, subsequently making cleaning difficult. AM was detached from the centre of the placenta, by cutting around the base of the umbilical cord, and then placed in chilled sterile saline (0.9% (w/v) NaCl), or Phosphate buffered saline (0.1M PBS), ready for washing.

To clean and remove all visible traces of contaminating blood, membranes were washed repeatedly, with sterile saline, in a sterile polypropylene tray (24 cm×30 cm×4 cm), for 20 minutes. Any visible blood contamination was removed by gently rubbing with fingertips. Larger clumps of congealed blood, and heavily stained membrane were removed using forceps and even by cutting with a No. 22 scalpel blade. Excess spongy layer lifted during rubbing was removed.

Using a No. 22 scalpel blade, the membranes were cut into 5-10 cm² segments followed by washing sequentially, for 5 minutes, in 4% (v/v), 8% (v/v) and 12% (v/v) dimethyl sulfoxide (DMSO, Sigma) in 100 mM PBS, respectively. At this point segments of fresh amniotic membrane and chorion were retained for protein and RNA analysis. Remaining segments were placed in 10 ml of 10 %(v/v) DMSO in PBS containing antibiotics (160 mg/L Gentamicin, 500 mg/L Cefuroxime) and then stored at −80° C. for a period of six months. The membranes were only used if the repeat virology screening excluded the specified diseases from the donor.

### Preparation of AM for Transplantation.

AM segments were thawed. Storage medium was removed and retained for analysis. To remove any residual storage DMSO, membrane segments were washed, 3 times, for 10 minutes in 5 ml sterile saline containing 1× protease inhibitors (Roche), with frequent vortexing. Each wash was labelled and retained for protein analysis. Membranes were now considered transplant ready. Washes were stored at −80° C. ready for protein analysis.

### Preparation of AM Storage and Wash Media for Protein Analysis.

Respective storage and wash media corresponding to a prepared membrane were either concentrated independently, concentrated as pooled washes and independent storage medium, or as a total media pool. For protein analysis of the storage medium and washes individually, samples were concentrated from 5 ml to 0.5 ml (10×) by concentration/buffer exchange (3,000 MWCO PES columns, Vivascience), in a 20 ml column at 4,000 g, at 4° C. Protein solubilisation was performed by addition of 3 ml extraction buffer to the concentrated samples, vortexed frequently for 45 min at room temperature, followed by re-concentration to 500 μl. Solubilisation of proteins prior to removal from the column improved overall protein recovery, solubilising any proteins that had become associated with the membrane. Sample concentration to volumes smaller than 500 μl was carried out using a 500 μl concentration/buffer exchange column (3,000 MWCO PES columns, Vivascience) at 12,000 g until the minimum volume of 20-50 μl was achieved. 450 μl 1× IEF extraction buffer was added to the column, vortexed, and incubated at room temperature for 30 min, with frequent vortexing, resulting in a final concentration 1/10^{th} the original sample volume. Insoluble cellular debris was removed by centrifugation at 21,000 g for 45 min, and then the protein concentration was determined (2-D Quant kit, Armersham Biosciences) according to the manufacturers protocol. Aliquots of 100 μl were stored at −80° C.

Concentration of pooled washes and master pools was carried out in a similar manor using a 20 ml column only. Columns were balanced using sterile saline containing 1× protease inhibitors (Roche) to a maximum volume of 20 ml. Concentration was carried out at 4,000 g for approximately 3 hours until a final volume of 0.5 ml was achieved. Columns were vortexed briefly to release any protein sediment, solubilised in 2 ml 1.1× IEF extraction buffer for 30 minutes, and then re-concentrated to 0.5 ml-1 ml. Insoluble cellular debris was removed by centrifugation at 21,000 g for 45 min, and then the protein concentration was determined (2-D Quant kit, Amersham Biosciences) according to the manufacturers protocol. Aliquots of 100 μl were stored at −20° C.

### 1-D SDS-PAGE

1-D SDS PAGE was performed using the Novex XCell SureLock mini system (Invitrogen), according to the manufacturer's protocol. Solubilised protein samples were prepared in a 1× loading buffer prepared from 4× NuPAGE LDS sample buffer, and 10× NuPAGE reducing agent (all Invitrogen) and deionised water to a maximum volume of 20 μl. Up to 11 samples were heat denatured at 90° C. for 5 min followed by loading on a 12 well NuPAGE Novex 4-12% Bis-Tris gel (Invitrogen) with 10 μl Multimark multi coloured standard (Invitrogen) in the reference well. Sample separation was performed using 1× SDS MES buffer (Invitrogen) at 200V for 40 min.

### Protein Visualisation

Protein visualisation was performed using Coomassie blue staining (Simply Blue safe stain, Invitrogen), microwave method, according to manufacturer's protocol. (FIG. 2)

### Western Blots

Western blots were carried out using the Novex XCell SureLock for the 1-D SDS-PAGE, described above, followed by Novex XCell II Blot module (Invitrogen) for protein transfer. 1 litre 1× transfer buffer was prepared from 50 ml 20× NuPAGE transfer buffer (Invitrogen), 200 ml methanol and 750 ml ultrapure water. Sponges and blotting paper were thoroughly pre-wetted with 1× NuPAGE transfer buffer. Sponges were squeezed repeatedly to ensure complete removal of air bubbles. Immobilon Psq PVDF (polyvinylidene fluoride, Millipore, Watford, UK) membranes were wetted using 50 ml methanol; gradually from one edge ensuring no air bubbles were introduced, and then placed in 1× transfer buffer.

### Immunodetection

Immunodetection was performed according to a standard protocol, using 9 cm² staining trays (Invitrogen) and rocking at 60 rpm, at room temperature (unless otherwise stated), detailed below. Buffers used were TBST (Tris buffered saline (Sigma), 0.05% (v/v) Tween 20 (Promega, Southampton, UK)), and TBSTM (TBST, 1% non-fat milk powder (Marval)).

### TGFb Immunodetection Using Western Blots

Detection of human TGF-β1 was carried out using monoclonal mouse anti-human TGF-β1 (MCA797, clone TB21; Serotec) primary antibody. TB21 can react with monomeric (12.5 kDa) or dimeric recombinant TGF-β1 under reducing and non-reducing conditions. Primary antibody was detected using alkaline-phosphatase conjugated goat anti-mouse IgG (H+L), (pre-adsorbed to bovine, horse, human antibodies, Pierce, Cheshire, UK). Blots were developed with premixed BCIP/NBT (Sigma). Protein extracted from platelets was used as the positive control for TGF-β1 antibody reactivity.

### Example 2

### Preparation of a Substantially Growth Factor Free AM

### Modified Procedure for Amniotic Membrane Preparation and Storage.

The following procedure was developed to isolate the placental AM and the reflectum AM separately and to optimise the removal of contaminating blood and excess spongy layer.

Immediately after delivery, umbilical cord clamps were applied to the end and base of the cord, to prevent excessive blood leakage onto the AM, thus minimising contamination. Before separating the AM from the chorion, intact reflectum foetal membrane was isolated from the placenta by cutting around the periphery of the placental body and then placed in chilled sterile saline (0.9% (w/v) NaCl). Placental AM was washed briefly with sterile saline, removing any surface contaminating blood, and then the AM was separated from the placenta, starting from one edge working inward, towards the cord. AM was detached from the base of the cord and immediately washed in sterile saline. Intact reflectum foetal membrane, consisting of amnion and chorion, was washed twice in sterile saline to remove contaminating blood and then the AM was separated from the chorion, as described previously, and placed in fresh sterile saline. No attempt was made to remove any contaminating blood from the AM. Both segments of AM (placental and reflectum) were washed three times in excess chilled sterile saline for 25 minutes on a rocker, 60 rpm. Once washed, only occasional and small amount of visible blood remained on the AM. In addition, prolonged washing and the lack of mechanical rubbing allowed the intact spongy layer to swell to three to four times its normal thickness. Washed AM was removed, spread on sterile plastic tray spongy side up, and any remaining visible spots of blood were removed.

A significant modification of the standard procedure was the removal of the spongy layer prior to storage. Removing the spongy layer was significantly easier after prolonged washing, rather than before. The excessive swelling enabled easy removal of the spongy layer, almost intact. This was performed using the reverse edge of a scalpel blade (No. 22). Starting at one edge of the AM, the spongy layer was gently lifted, lifting it away from the AM. Once lifted this was used to pull the layer from the AM. This was performed across the whole membrane, separating the spongy layer in its entirety. A brief wash in fresh saline was performed to remove any loose debris. Removing the spongy layer from the AM removed any remaining contaminating blood from the amnion leaving a white and translucent membrane. The removed spongy layer was retained and stored at −80° C. for further analysis. Cleaned membranes were prepared for storage as described above.

### Combined Results for Example 1 and 2.

To confirm TGF-β1 protein in AM, crude protein extracted from fresh AM, TRAM, and spongy layer removed during processing, SDS-PAGE analysis was performed using equivalent loadings of each sample. (FIG. 2a). Western blots were then carried out with anti-human TGF-β1 being used to detect the protein (FIG. 2b).

TGF-β1 protein was detected in all fresh AM samples, however, at varying intensities (FIG. 2). These initial results suggested considerable inter-membrane variation. On the other hand, TGF-β1 was detected in only one of the corresponding processed AM samples, indicating preservation and processing removes TGF-β1 in some cases. The most intense staining was observed in the spongy layer removed during processing, so much so that in some samples staining appeared smudged across 2-3 lanes.

Immediately, our results intimated that TGF-β1 content of fresh AM was variable between membranes. In addition, despite removal of TGF-β1 during processing, certain membranes retained detectable levels, which in a clinical environment would be transplanted to the eye.

Protein was extracted from seventeen AM's at several points during the processing and preservation procedure, and during preparation for transplantation. At the same time, corresponding samples from fresh (un-preserved) chorion, and spongy layer removed during processing prior to preservation were also collected.

Western blotting followed by immuno-detection of TGF-β1 using anti-human TGF-β1 antibody was carried out. Typical examples demonstrating effect of processing resulting in variable TGF-β1 elution are shown (FIG. 4.1), and total sample population are given (Table 1).

Staining was most intense in samples prepared from the spongy layer and chorion, varying considerably between membranes. Although fresh AM stained intense when crude extracts were used (FIG. 2), the relative staining of fresh AM in the load-balanced blots, appeared low (FIG. 4.1). This suggested the spongy layer and chorion as considerable sources of TGF-β1.

Intense staining was observed in storage medium (FIG. 4.1), suggesting initial TGF-β1 release occurred because of the preservation process. To support this, TGF-β1 was not detected in storage media used to wash membranes prior to preservation. Staining of TGF-β1 in the storage and wash media appeared more intense than in fresh AM. This was an effect of concentrating (10× ) the media to achieve equivalent protein loads across all samples. In doing so, this demonstrated that TGF-β1 released during processing generally decreased with each sequential wash, until no longer detected (FIG. 4.1). This typically occurred within three 10ml saline washes of 5 minutes per wash.

Variation in TGF-β1 content of fresh membranes (FIG. 4.1, A and B), affected the relative amount released during processing (FIG. 4.1, lane 9), and the amount of processing required until detectable TGF-β1 was no longer eluted (FIG. 4.1, lanes A8 and B7). Where the spongy layer had not been previously removed, however, significant numbers of washes were required in order to remove sufficient TGF-β1 to be below a detectable level, with a 20 μg protein load.

Referring to FIG. 4.2, there is shown titration of human TGF-β1 purified form platelets to establish the levels of TGF-β1 in AM and spongy layer. TGF-β1 purified from platelets (RnD, UK) was titrated from 200 ng down to 3.1 ng and the respective amounts were loaded in each well. Proteins were separated on denaturing PAGE mini-gels under reducing conditions; western blotted to PVDF and detected with anti-TGF-β1 antibody. Lanes shown are 200 ng, 100 ng, 50 ng, 12.5 ng, 6.25 ng, and 3.1 ng. Positive staining is indicated by the arrows, and was down to 50 ng. The staining intensity of the spongy layer (FIG. 4.1) was at least 4 fold greater than that observed for 200 ng human TGF-β1 load;

However, TGF-β1 was still detected in subsequent washes (FIG. 5, lane 7) in one in five membranes. In these cases, elution appeared much slower and continuous, resulting in similar staining intensities in the storage medium and each sequential wash (FIG. 5, lanes 4-7). In addition, the total relative amount released (measured by the relative staining intensity of the pooled washes) in the first three (standard) washes appeared to be appreciably less than in other membranes (FIG. 5, lane 8). In these samples, TGF-β1 was often detected in the fifth wash (data not shown) and in the membrane after washing. In addition, staining in spongy layer was noticeably more intense, suggesting the spongy layer could also be acting as an additional TGF-β1 source, prolonging elution into the washes.

It is noteworthy that the processing procedure after preservation was standardised for all membranes to produce comparative data. In doing so, processing was considerably more thorough than procedures used clinically. Despite this, TGF-β1 elution varied, often continuing beyond 20 minutes of extensive washing with agitation. Clinical preparation procedures (two brief rinses in physiological saline) would therefore introduce greater variation, resulting in greater amounts of TGF-β1 remaining in the AM.

### Example 3

### Immunohistochemical Analysis of Amniotic Membrane, and Spongy Layer

### Immunohistochemistry

### General Reagents

TBST—Tris buffered saline (TBS; Sigma) with 0.05% Tween 20 (Promega, UK) was used to prepare all immunohistochenical solutions and buffers. When dissolved in ultrapure water, 1 sachet prepared 1 litre of 50 mM Tris, 138 mM NaCl, 27 mM KCl, pH 8 at 25° C. For immunohistochemistry, the pH was adjusted to 7.6 using concentrated HCl. Finally Tween 20 was added 0.5 μl/ml to a final concentration of 0.05% (v/v).

3-aminopropyltriethoxysilane-3-aminopropyltriethoxysilane (APES; Sigma) was used to coat slides to improve tissue adhesion to the slide during staining. To APES coat slides, slides were sequentially immersed in acetone for 20 seconds, freshly prepared 2% APES in acetone for 20-30 seconds, running tap water for 30 seconds and finally rinsed in ultrapure water before being dried overnight at room temperature. Dry slides were use the next day or stored at room temperature.

### Optimum Cutting Temperature Compound

Optimum Cutting Temperature compound (OCT) (Dako, Ely, UK) was used to provide a medium to embed tissue samples when preparing frozen sections by snap freezing in liquid nitrogen.

### Immunohistochemicals.

### Blocking Reagent

DAKO protein Block (DAKO) was used as a non-specific blocking agent on tissue sections before the application of the primary antibody.

### Primary Antibodies

### TGF-β1

Mouse monoclonal anti-human TGF-β1, clone TB21 was obtained from Serotec, Oxford, UK and was stored at 4° C. until required. A titration assay established the optimum antibody dilution of 1:20 when APAAP methods were employed.

### Anti-Human LAP (TGF-β1) Antibody

Monoclonal mouse anti-human LAP (TGF-β1) antibody (clone 27235.1) was obtained from R&D systems, Oxon, UK, and was stored at 4° C. until required. Specificity is for human recombinant LAP and natural LAP. Recommended use is 1-2 μg/ml, however, a titration assay established the optimum antibody dilution of 1:100 (5 μg/ml) when APAAP methods were employed.

### Latent TGF-β1 Binding Protein I Antibody

Monoclonal mouse anti-human Latent TGF-β Binding Protein 1 antibody (clone 35409) was obtained from R&D systems, UK, and was stored at 4° C. until required. Specificity is for human LTBP-1. A titration assay established the optimum antibody dilution of 1:100 (5 μg/ml) when APAAP methods were employed.

### Secondary Antibody

Rabbit anti-mouse antibody (Z0259) was obtained from DAKO, UK, specifically for APAAP immunohistochemistry procedures. Antibody was used at a dilution of 1:40.

### Tertiary Antibody

Calf intestinal Alkaline Phosphatase and mouse monoclonal Anti-Alkaline Phosphatase (APAAP) was obtained from DAKO, UK, and stored at 4° C. until required. The antibody was used at a working stock of 1:40, diluted in TBST.

### General Immunohistochemical Reagents

### Fast Red

Fast red kit (Sigma) was the substrate-chromogen for streptavidin-biotin-APAAP HRP technique. The kit contained tris buffer tablets and fast red tablets (0.6 mM levamisole to block endogenous alkaline phosphatase activity), which were stored at −20° C. until required. For use, the solution was freshly prepared by sequentially dissolving one tablet of tris buffer and one Fast Red tablet in 1 ml ultapure water. Before use, the solution was centrifuged at 21,000 g for 1 min to pellet any fast red particulates. Particulate carryover otherwise resulted in background speckles in the slides.

### Haematoxylin

Slides were immersed sequentially in Haematoxylin (Heamalum Mayers; Nustain, Nottingham, UK) stain for 10-30 seconds, in tap water for 10 seconds, followed by ammonium solution (375 mM). Excess water was removed from the slide using a tissue (Care was taken not to touch the section), and a cover slip applied and allowed to fix. Slides were then examined under a microscope at 40×, 100×, and 400× powers of magnification.

### Glycergel

Glycergel (Dako) was an aqueous mounting medium stored at 4° C. until required. For use, the solution was warmed to 45° C., and the solution applied drop wise to each slide to secure the cover slip. Care was taken not to introduce air bubbles.

### Procedure

Immunohistochemistry was performed on sections of frozen embedded amniotic membrane segments, using antibodies against various markers of interest in the presence of appropriate positive (where possible) and negative controls. Antibodies were initially titrated in order to determine the optimum dilution for use with each specific sample. For example, the TGF-β1 antibody was used in dilutions of 1:10 to 1:100, determining the titre at which the optimum staining pattern obtained to be 1:20; therefore, this was used as the working dilution.

### Sample Freezing

AM samples were obtained fresh, during processing for preservation, or transplant ready, obtained after preservation and processing. Prior to tissue freezing, foil cups (20 mm deep) formed around the base of a Bijou tube were filled half way with OCT, and pre-chilled in nitrogen vapour to thicken the OCT, but not solidify it (this was to aid tissue positioning for freezing). AM segments (2 cm×4 cm) were covered in OCT in a petri dish, and positioned spongy side down on a dry lens tissue. The lens tissue provided support, but did not to interfere with sectioning, and did not stain during immunodetection procedures. Segments were gently rolled until 8-10 mm diameter was obtained. Using a No. 22 blade, 10 mm lengths were carefully cut, immersed end-on into semi-solid OCT, and additional OCT was used to cover the tissue and fill the cup. Care was taken not to introduce air bubbles or disrupt the roll, as sectioning would be affected. Cups were place on a raft in nitrogen until frozen. Immersing the cup directly in liquid nitrogen caused fracturing of the OCT and tissue. Once frozen, OCT blocks were wrapped in parafilm, placed in a sealable bag with a slight cut to allow air to escape, and then placed in liquid nitrogen to ensure complete freezing. Samples were stored at −80° C., until sections were prepared.

### Preparing Sections

Samples were placed in the cryostat (−20° C.) at least 1 hour before sectioning to equilibrate temperature, preventing fracturing of the OCT block, during sectioning. Foil was completely removed to prevent damage to the cutting blade, the sample secured onto the pre-chilled block end-on using OCT, and placed in the chuck set to the home position. The block was trimmed until a smooth cutting surface was obtained, following which 6 μ sections were cut. Varying the position of the cover slide during sectioning prevented the section from crumpling and tearing. Sections were mounted (two sections per slide) on APES coated slides. Slides were air dried ready for fixing or immediately hematoxylin stained to assess sectioning.

### Fixing Slides

Before fixing, the slides were air dried over night to allow all water to evaporate. Slides were immersed in pre-chilled (4° C.) acetone for 5-10 minutes, and placed at room temperature to allow acetone evaporation and slides to dry. Slides were stained immediately or placed into a slide rack, wrapped in foil, and stored at −20° C. until needed.

### Immunostaining Using the APAAP Method.

Immunostaining of slides with specific antibodies was performed according to a standard departmental protocol for APAAP. Slides were immersed in TBST for five minutes, three times. Excess liquid was removed from around the specimens followed by the addition of blocking agent (100 μl per section) for 30-60 minutes. Excess block was removed by tapping the slide (not washing). The primary monoclonal antibody was diluted appropriately in TBST. 100 μl was added to each section, and incubated for 30-45 minutes at room temperature in a moist chamber. It was essential that the slides did not dry out at any point, as this affected the end result. Slides were washed twice in TBST for 5 minutes, followed by the removal of excess liquid. The 2° antibody was diluted appropriately in TBST. 100 μl of the diluted 2° antibody was applied to each section, and incubated for 20-30 minutes at room temperature in a moist chamber to prevent slides from drying. During the incubation period the substrate-chromagen Fast Red was prepared. The slides were washed again in TBST twice for five minutes and then incubated with 100 μl per section steptavidin-biotin alkaline phosphatase anti alkaline phosphatase (APAAP) complex for 30 minutes TBST washing of the slides was repeated, followed by the removal of excess liquid. To each section 100 μl of prepared substrate-chromogen was added. Slides were repeatedly examined under a light microscope and staining continued until the desired staining was achieved (usually between 2-10 minutes). Positive staining was indicated by a red colour. The slides were rinsed in ultra pure water, terminating the staining reaction, and were then counter stained with Hematoxylin. Cover slides were secured over the sections and the slides examined under a light microscope.

### Immunohistochemical Analysis of AM.

Preservation and processing results in elution of TGF-β1 from AM; however, despite prolonged washing, TGF-β1 often remain detectable at low levels. TGF-β1 in situ localisation and the relative effects of processing and release, were therefore determined.

Immunohistochemical analysis of eight fresh and processed AM was carried. Sections were made from frozen samples of fresh AM and corresponding AM preserved and processed for transplantation. Immunohistochemistry for anti-TGF-β1, anti-β1-LAP, and anti-LTBP was performed using the APAAP procedure described in the methods and material. Control staining with non-immune IgG was negative (data not shown).

Staining for TGF-β1 varied between membranes depending on the morphology of the membrane. Typically, two morphologically distinct classification of AM were observed, which were termed “thin” and “thick” membranes. Thin membranes were composed of an epithelial monolayer, supported by a thin ECM. The ECM was sparsely populated by a fibroblast monolayer on the basal edge of the membrane, adjacent to the spongy layer interface. Thick membranes differed in that the ECM was considerably thicker, and was populated throughout by additional fibroblasts, organised in multilayered fashion. A recent report described thickening of the amnion basement membrane in response to inflammatory cytokines produced during placental abnormalities, maternal and foetal disorders[42].

Typically, fresh thick AM stained intensely for TGF-β1 throughout the entire membrane. Due to this, localisation of TGF-β1 to specific structures proved difficult; however, the spongy layer stained particularly intensely. On the other hand, in thin fresh membranes, intense TGF-β1 staining was localised in the ECM around fibroblasts, and as a distinct line in the BM beneath the AEC, but as in thick membranes, staining was most intense in the spongy layer. Preservation and processing reduced TGF-β1 staining in all membranes, particularly in thick membranes. Reduced staining is therefore indicative that TGF-β1 is soluble which is then eluted during processing. This suggests that some membranes contain greater amounts of soluble TGF-β1, which typically appears as non-localised general staining. This was especially the case in the spongy layer, which stained intensely in fresh membranes, but was reduced the most through processing (FIG. 6).

Staining for TGF-β1 after preservation and processing was similar for all membranes, specifically localised in the ECM around fibroblasts and immediately basal to AEC. Increased fibroblast numbers in thick membranes resulted in increased staining, suggesting that thick membranes would retain more TGF-β1. Similar staining intensity and localisation before and after processing suggest TGF-β1 was ECM-associated, which was not affected by processing. Occasionally, punctuated TGF-β1 staining was observed in AEC of fresh AM and in corresponding TRAM, which was not reduced by processing (FIG. 6).

These results indicate that TGF-β1 exists in at least two forms in AM. General staining is suggestive of a soluble form, which is reduced after processing, whilst TGF-β1 localised to specific regions of the AM is present as an insoluble bound form. This indicates that TGF-β1 activity in AM varies between membranes at the point of preservation (FIG. 6).

Referring to FIG. 10, the identification of thrombospondin-1 (TSP-1; Spots 21, and 62) in TRAM confirms TSP-1 expression in amnion. TSP-1 participates in cell-to-cell and cell-to-matrix communication[43], and has been implicated in the mediation of cellular adhesion, proliferation, differentiation and migration, and also apoptosis [44]. More importantly, TSP-1 is reported to control a number of physiological processes such as wound repair, inflammatory response, and angiogenesis [43, 45]. TSP-1 was detected and fresh AM, and spongy layer. The procedure to remove the spongy layer reduced the levels of TSP-1 detected in preserved AM, with TSP-1 being detected in isolated spongy layer (as shown in FIG. 10).

### Example 4

### 2D Electrophoresis of Proteins from the Amniotic Membrane and from the Spongy Layer

### 2-Dimensional Electrophoresis

Standard 2-D gel electrophoresis was used with replicate gels being performed (within the same batch) to eliminate technical variation, which can cause deviations in the number of spots detected between gels thus ensuring the 2-D pattern is valid. Gels were stained using a modified Yan and Wait mass spectrometry compatible silver staining protocol.

### Reagents

Fixative; 40% (v/v) methanol, 10% (v/v) acetic acid (Sigma); Sensitiser; 40% (v/v) methanol, 68% (w/v) sodium acetate (Sigma) and 0.2% (w/v) sodium thiosulphate (Sigma); Stop; 15%(w/v) EDTA (Sigma); Impregnating solution; 2.5% (w/v) silver nitrate (Fisher) (prepared and stored in the Dark); Developer, 25% (w/v) sodium carbonate (Sigma), 0.4% (w/v) formaldehyde (Sigma) (prepared minus the reductant and chilled to 4° C. Formaldehyde addition was immediately before use).

### Protocol:

Fix for 2×15 minutes; Sensitize for 30 minutes; Wash for a minimum of 3×5 minutes; Impregnate for 20 minutes; wash for 2×1 minute; Develop; Stop for 10 minutes;

Imaging gels

The 2-D gels were digitised using the GS-800 from Bio-Rad, according to manufacturers protocols. The calibrated digitised images were 93.5 microns (pixal diameter) and were saved as 10 Mb files. Gels were analysised using Delta 2D.

### Statistics

Inter-membrane variation was assessed using the Friedman test (Nonparametric test), and Dunn's multiple comparison test to determine any statistical variation between membranes.

### Results

Comparing the protein profiles of transplant ready amniotic membrane, minus spongy layer, demonstrated that variation between membrane preparations was not significant (P=0.0639) (FIG. 7). In addition, Dunn's multiple comparison test showed that individually paired replicate groups were not significantly different (P>0.05).

Reference to FIG. 8 confirms the presence of a significant number of proteins in the spongy layer. By generating a composite of 2 DE gels (FIG. 9), one of TRAM proteins and one of matched spongy layer proteins, it is possible to identify those proteins that are associated with both structures and those that reside solely in either the AM or the spongy layer. This shows that although the spongy layer contains proteins similar to AM (possibly carryover/cellular contamination), it also contains many proteins not detectable in the AM. Thus, the removal of the spongy layer from the AM removes a significant amount of protein, leaving behind a less protein-rich scaffold to support the migration of epithelial cells.

### REFERENCES

1. Davis, J. W., “Skin transplantation with a review of 550 cases at the Johns Hopkins Hospital.” Johns Hopkins Medical Journal, 1910; 15: 307.
2. Arora, M., K. A. Jaroudi, C. J. Hamilton, and F. Dayel, “Controlled comparison of interceed and amniotic membrane graft in the prevention of postoperative adhesions in the rabbit uterine horn model”. Eur J Obstet Gynecol Reprod Biol, 1994; 55(3): 179-82.
3. Colocho, G., W. P. Graham, 3rd, A. E. Greene, D. W. Matheson, and D. Lynch, “Human amniotic membrane as a physiologic wound dressing”. Arch Surg, 1974; 109(3): 370-3.
4. DeRoth, A., “Plastic repair of conjunctival defects with fetal membrane.” Arch Ophthalmol, 1940; 23: 522.
5. Dhall, K., “Amnion graft for treatment of congenital absence of the vagina”. Br J Obstet Gynaecol, 1984; 91(3): 279-82.
6. Faulk, W. P., R. Matthews, P. J. Stevens, et al., “Human amnion as an adjunct in wound healing”. Lancet, 1980; 1(8179): 1156-8.
7. Gruss, J. S. and D. W. Jirsch, “Human amniotic membrane: a versatile wound dressing”. Can Med Assoc J, 1978; 118(10): 1237-46.
8. Nisolle, M. and J. Donnez, “Vaginoplasty using amniotic membranes in cases of vaginal agenesis or after vaginectomy”. J Gynecol Surg, 1992; 8(1): 25-30.
9. Prasad, J. K., I. Feller, and P. D. Thomson, “Use of amnion for the treatment of Stevens-Johnson syndrome”. J Trauma, 1986; 26(10): 945-6.
10. Rennekampff, H. O., P. Dohrmann, R. Fory, and F. Fandrich, “Evaluation of amniotic membrane as adhesion prophylaxis in a novel surgical gastroschisis model”. J Invest Surg, 1994; 7(3): 187-93.
11. Sorsby, A. and H. M. Symmons, “Amniotic membrane grafts in caustic burns of the eye (bums of second degree)”. Br J Ophthalmol, 1946; 30: 337-45.
12. Subrahmanyam, M., “Amniotic membrane as a cover for microskin grafts”. Br J Plast Surg, 1995; 48(7): 477-8.
13. Trelford, J. D. and M. Trelford-Sauder, “The amnion in surgery, past and present”. Am J Obstet Gynecol, 1979; 134(7): 833-45.
14. Troensegaard-Hansen, E., “Amniotic grafts in chronic skin ulceration”. Lancet, 1950; 1: 859-60.
15. Ward, D. J. and J. P. Bennett, “The long-term results of the use of human amnion in the treatment of leg ulcers”. Br J Plast Surg, 1984; 37(2): 191-3.
16. Ward, D. J., J. P. Bennett, H. Burgos, and J. Fabre, “The healing of chronic venous leg ulcers with prepared human amnion”. Br J Plast Surg, 1989; 42(4): 463-7.
17. Sheridan, R. L. and C. Moreno, “Skin substitutes in burns”. Burns, 2001; 27(1): 92.
18. Reim, M., C. Redbrake, and N. Schrage, “Chemical and thermal injuries of the eyes. Surgical and medical treatment based on clinical and pathophysiological findings”. Arch Soc Esp Oftalmol, 2001; 76(2): 103-24.
19. Chatterjee, S. R., “Two case reports on vaginoplasty in cases of congenital absence of vagina”. J Indian Med Assoc, 2003; 101(10): 606-7.
20. Samandari, M. H., M. Yaghmaei, M. Ejlali, M. Moshref, and A. S. Saffar, “Use of amnion as a graft material in vestibuloplasty: a preliminary report”. Oral Surg Oral Med Oral Pathol Oral Radiol Endod, 2004; 97(5): 574-8.
21. Arnold, P. B., C. W. Green, P. A. Foresman, and G. T. Rodeheaver, “Evaluation of resorbable barriers for preventing surgical adhesions”. Fertility and Sterility, 2000; 73(1): 157-161.
22. Lavery, F., S., “Lime burn of conjunctiva and cornea treated with amnioplastic graft”. Trans Ophthalmol Soc UK, 1946; 66: 668.
23. Adinolfi, M., C. A. Akle, I. McColl, et al., “Expression of HLA antigens, beta 2-microglobulin and enzymes by human amniotic epithelial cells”. Nature, 1982; 295(5847): 325-7.
24. Houlihan, J. M., P. A. Biro, H. M. Harper, H. J. Jenkinson, and C. H. Holmes, “The human amnion is a site of MHC class Ib expression: evidence for the expression of HLA-E and HLA-G”. J Immunol, 1995; 154(11): 5665-74.
25. Akle, C. A., M. Adinolfi, K. I. Welsh, S. Leibowitz, and I. McColl, “Immunogenicity of human amniotic epithelial cells after transplantation into volunteers”. Lancet, 1981; 2(8254): 1003-5.
26. Tseng, S. C., P. Prabhasawat, and S. H. Lee, “Amniotic membrane transplantation for conjunctival surface reconstruction”. Am J Ophthalmol, 1997; 124(6): 765-74.
27. Terranova, V. P. and R. M. Lyall, “Chemotaxis of human gingival epithelial cells to laminin. A mechanism for epithelial cell apical migration”. J Periodontol, 1986; 57(5): 311-7.
28. Khodadout, A. A., A. M. Silverstain, and K. R. Kenyon, “Adhesion of regernerating corneal epithelium. The role of the basement membrane.” Am J Ophthalmol, 1968; 65: 339-48.
29. Kurpakus, M. A., E. L. Stock, and J. C. Jones, “The role of the basement membrane in differential expression of keratin proteins in epithelial cells”. Dev Biol, 1992; 150(2): 243-55.
30. Guo, M. and F. Grinnell, “Basement membrane and human epidermal differentiation in vitro”. J Invest Dermatol, 1989; 93(3): 372-8.
31. Boudreau, N., C. J. Sympson, Z. Werb, and M. J. Bissell, “Suppression of ICE and apoptosis in mammary epithelial cells by extracellular matrix”. Science, 1995; 267(5199): 891-3.
32. Tseng, S. C., P. Prabhasawat, K. Barton, T. Gray, and D. Meller, “Amniotic membrane transplantation with or without limbal allografts for corneal surface reconstruction in patients with limbal stem cell deficiency”. Arch Ophthalmol, 1998; 116(4): 431-41.
33. Shimazaki, J., N. Shinozaki, and K. Tsubota, “Transplantation of amniotic membrane and limbal autograft for patients with recurrent pterygium associated with symblepharon”. Br J Ophthalmol, 1998; 82(3): 235-40.
34. Sato, H., J. Shimazaki, N. Shimazaki, and e. al., “Role of growth factors for ocular surface reconstruction after amniotic membrane transplantation.” Invest Ophthalmol Vis Sci, 1998; 39: S428.
35. Lee, S. B., D. Q. Li, D. T. Tan, D. C. Meller, and S. C. Tseng, “Suppression of TGF-beta signaling in both normal conjunctival fibroblasts and pterygial body fibroblasts by amniotic membrane”. Curr Eye Res, 2000; 20(4): 325-34.
36. Tseng, S. C., D. Q. Li, and X. Ma, “Suppression of transforming growth factor-beta isoforms, TGF-beta receptor type II, and myofibroblast differentiation in cultured human corneal and limbal fibroblasts by amniotic membrane matrix”. J Cell Physiol, 1999; 179(3): 325-35.
37. Li, D. Q. and S. C. Tseng, “Three patterns of cytokine expression potentially involved in epithelial-fibroblast interactions of human ocular surface”. J Cell Physiol, 1995; 163(1): 61-79.
38. Dua, B. S., J. A. Gomes, A. J. King, and V. S. Maharajan, “The amniotic membrane in ophthalmology”. Surv Ophthalmol, 2004; 49(1): 51-77.
39. Saharinen, J., M. Hyytiainen, J. Taipale, and J. Keski-Oja, “Latent transforming growth factor-beta binding proteins (LTBPs)—structural extracellular matrix proteins for targeting TGF-beta action”. Cytokine & Growth Factor Reviews, 1999; 10(2): 99-117.
40. Border, W. A. and N. A. Noble, “Transforming growth factor beta in tissue fibrosis”. N Engl J Med, 1994; 331(19): 1286-92.
41. Tsubota, K. and J. Shimazaki, “Surgical treatment of children blinded by Stevens-Johnson syndrome”. Am J Ophthalmol, 1999; 128(5): 573-81.
42. Castro, E. C., M. A. Reis, and V. P. Teixeira, “Thickening of the amnion basement membrane and its relationship to placental inflammatory lesions and fetal and maternal disorders”. Eur J Obstet Gynecol Reprod Biol, 2004; 114(2): 171-6.
43. Bourne, G. L., “The microscopic anatomy of the human amnion and chorion”. Am J Obstet Gynecol, 1960; 79: 1070-3.

1. A method of preparing substantially growth factor free amniotic membrane (GFF-membrane), which method comprises the steps of:
(a) isolating amniochorionic membrane from placenta; and
(b) removing a chorion membrane and spongy layer from the amniochorionic membrane, to thereby produce substantially growth factor free (GFF) amniotic membrane.

2. A method according to claim 1, wherein the method reduces the concentration of growth factors in the amniotic membrane by at least 55% (w/w) when compared to normal amniotic membrane containing the spongy layer.

3. A method according to claim 1, wherein after step (a) but before step (b), the method comprises a step of washing the amniochorionic membrane to remove any excess biological fluids derived from the mother.

4. A method according to claim 3, wherein the washing step is conducted in sterile solution.

5. A method according to claim 4, wherein the sterile solution comprises 0.7-1.2% (w/v) NaCl, and the washing step is carried out for at least 10 minutes.

6. A method according to claim 1, wherein after step (a) but before step (b), the method comprises an additional step of separating the amniochorionic membrane into amniotic membrane and chorion membrane.

7. A method according to claim 1, wherein after step (a) but before step (b), the method comprises a step of soaking the amniotic membrane in a sterile solution capable of loosening the connection between various layers in the amniotic membrane for sufficient time to enable subsequent removal of the spongy layer therefrom in step (b).

8. A method according to claim 7, wherein the soaking step is carried out in saline, which may be physiological saline or phosphate buffered saline (PBS).

9. A method according to claim 8, wherein the saline solution comprises 0.7-1.2% (w/v) NaCl, and the soaking step is carried out for at least 10 minutes.

10. A method according to claim 7, wherein the soaking step is carried out several times until blood contamination has been eliminated.

11. A method according to claim 1, wherein after step (b), once the spongy layer has been removed, the amniotic membrane is washed in saline to remove residual spongy layer debris.

12. A method according to claim 1, wherein the method comprises a further step after step (b), which comprises preservation and/or storage of the prepared GFF-membrane.

13. A method according to claim 12, wherein the preservation step comprises contacting the amniotic membrane with a suitable preservation chemical.

14. A method according to claim 12, wherein the preservation step comprises a freezing step.

15. A method according claim 14, wherein after the freezing step, the method comprises a step of thawing the stored amniotic membrane to about room temperature, and then a step of washing the amniochorionic membrane to remove cellular debris therefrom.

16. A method according to claim 15, wherein the washing step is conducted in sterile solution.

17. A method according to claim 16, wherein the washing step is carried out for at least 10 minutes.

18. A method according to claim 17, wherein the washing step comprises at least two cycles of washes in saline for at least 10 minutes each cycle.

19. A method according to claim 15, wherein the method including the final washing step reduces the concentration of growth factors in the amniotic membrane by at least 85% (w/w), when compared to normal amniotic membrane containing the spongy layer.

20. A substantially growth factor free (GFF) amniotic membrane.

21. A substantially (GFF) amniotic membrane according to claim 20, wherein the GFF-membrane is produced by the method according to claim 1.

22. A method of preparing enriched amniotic membrane (E-membrane), which method comprises contacting a substantially growth factor-free (GFF) amniotic membrane with a membrane-enriching compound in conditions suitable to allow uptake of the compound by the GFF amniotic membrane to thereby produce enriched amniotic membrane.

23. A method according to claim 22, wherein the enrichment compound comprises a growth factor, steroid, hormone, antimicrobial agent, or any other beneficial molecule, or any desired compatible combination of the foregoing.

24. A method according to claim 23, wherein the growth factor comprises EGF, TGF-α, KGF, HGF, bFGF, NGF, TGF-β1, TGF-β2, TGF-β3, TSP-1, PEDF, or any combination thereof.

25. A method according to claim 23, wherein the steroid comprises Prednisolone phosphate, Prednisolone acetate, Betamethasone, or Dexamethasone.

26. A method according to claim 22, wherein the GFF amniotic membrane is produced by the method according to claim 1.

27. A method according to claim 22, wherein the contacting step comprises incubating the GFF amniotic membrane in a solution, which solution comprises the membrane-enriching compound, under conditions suitable for the compound to be absorbed by the amniotic membrane.

28. A method according to claim 22, wherein the method comprises a further step, which comprises preservation and/or storage of the E-membrane.

29. An enriched amniotic membrane (E-membrane) comprising at least one amniotic membrane-enriching compound present at a concentration greater than its corresponding concentration when in normal physiological conditions.

30. An E-membrane according to claim 29, wherein the enriched membrane is produced by the method according to claim 22.

31-33. (canceled)

34. A method of treating a subject suffering from a wound or fibrotic disorder, the method comprising administering to a subject in need of such treatment, a therapeutically effective amount of substantially growth factor free (GFF) amniotic membrane according to claim 20, or spongy layer or a component thereof isolated from amniotic membrane, or enriched amniotic membrane (E-membrane) according to claim 29.

35. A pharmaceutical composition comprising a therapeutically effective amount of substantially growth factor free (GFF) amniotic membrane according to claim 20, or spongy layer or a component thereof isolated from amniotic membrane, or enriched amniotic membrane (E-membrane) according to claim 29, and a pharmaceutically acceptable diluent, carrier or excipient.

36-37. (canceled)

38. A method of treating a subject suffering from an ophthalmological condition, the method comprising administering to a subject in need of such treatment, a therapeutically effective amount of substantially growth factor free (GFF) amniotic membrane according to claim 20, or spongy layer or a component thereof isolated from amniotic membrane, or enriched amniotic membrane (E-membrane) according to claim 29.

39. The method of claim 38, wherein the ophthalmological condition is selected from the group consisting of those characterised by a damaged ocular surface, chronic state of chemical and thermal bums, diseases of the eye, Persistent epithelial defects, Neurotrophic keratitis, Bullous Keratopathy, excision of lesions, excision of tumour of conjunctiva, stem cell transplant surgery, acute inflammation, acute state of chemical and thermal burns, corneal stromal melting diseases, Rheumatoid Keratopathy, Viral keratitis and bacterial ulcers.
